(19) United States
(12) Patent Application Publication
(10) Pub. No.: US 2016/0303245 A1
(43) Pub. Date: Oct. 20, 2016

(54) FATTY ACID DERIVATIVES OF DIMERIC INHIBITORS OF PSD-95

(71) Applicant: UNIVERSITY OF COPENHAGEN, Copenhagen K (DK)

(72) Inventors: Kristian Strømgaard, Roskilde (DK); Anders Bach, Valby (DK); Klaus Bertram Nissen, Årslev (DK)

(21) Appl. No.: 15/100,687

(22) PCT Filed: Nov. 26, 2014
(86) PCT No.: PCT/DK2014/050402

§ 371 Date: Jun. 1, 2016

(30) Foreign Application Priority Data
Dec. 1, 2013 (DK) PA 2013 70735

(51) Int. Cl. A61K 47/48; A61K 38/00

(52) U.S. Cl. CPC A61K 47/48038; A61K 38/00

(57) ABSTRACT

The present invention provides fatty acid derived compounds capable of binding to the PDZ domains of PSD-95 and their medical use as inhibitors of protein-protein interaction mediated by PSD-95.

### FIELD OF INVENTION

The present invention relates to compounds capable of binding to the PDZ domains of PSD-95 and their medical use as inhibitors of protein-protein interaction mediated by PSD-95.

### BACKGROUND OF THE INVENTION

Postsynaptic density protein-95 (PSD-95) is a protein encoded in humans by the DLG4 (disks large homolog 4) gene. PSD-95 is a member of the membrane-associated guanylate kinase (MAGUK) family and is together with PSD-93 recruited into the same NMDA receptor and potassium channel clusters.

PSD-95 is the best studied member of the MAGUK family of PDZ domain-containing proteins. Like all MAGUK family proteins, it includes three PDZ domains, an SH3 domain, and a guanylate kinase-like domain (GK) connected by linker regions. It is almost exclusively located in the postsynaptic density of neurons, and is involved in anchoring synaptic proteins. Its direct and indirect binding partners include neuroligin, neuronal nitric oxide synthase (nNOS), N-methyl-D-aspartate (NMDA) receptors, AMPA receptors, and potassium channels.

The PDZ domain is a common structural domain of 80-90 amino-acids predominantly found in scaffolding proteins of various organisms including humans. PDZ is an acronym for the first letters of three proteins—PSD-95, Drosophila disc large tumor suppressor (DIg1), and Zonula occludens-1 protein (ZO-1)—which were the first proteins discovered comprising the domain.

In general, PDZ domains interact with other proteins by binding to their C-terminus. This is achieved by β-sheet augmentation, meaning that the β-sheet in the PDZ domain is extended by the addition of the C-terminal tail of the binding partner protein and thus forming an extended β-sheet like structure.

PDZ domains are found in a wide range of proteins both in the eukaryotic and eubacteria kingdoms, whereas there are very few examples of the protein in archaea. The three PDZ domains of PSD-95, PDZ1-3, bind peptide ligands with similar consensus sequence such as Ser/Thr-X-Val/Ile/Leu-COOH.

The structural basis for the interaction of PDZ domains with C-terminal peptides was first elucidated by an X-ray crystallographic structure of PDZ3 of PSD-95 complexed with a native peptide ligand, CRIPT (Sequence: YKQTSV). PDZ3 contains six antiparallel β-strands (βA-βF) and two α-helices (αA and αB), and the C-terminal peptide ligand binds into a groove between the βB strand and αB helix. Two residues in the peptide ligand are considered particularly important for affinity and specificity, the first (P⁰) and the third (P⁻²) amino acids as counted from the C-terminus. The side chain of the amino acid in P⁰ position projects into a hydrophobic pocket and an amino acid with an aliphatic side chains (Val, Ile and Leu) is required. In the PDZ3-CRIPT X-ray crystal structure, the hydroxyl oxygen of Ser or Thr (P⁻²) forms a hydrogen bond with the nitrogen of an imidazole side chain of His372, and this interaction has been shown to be an important determinant for the affinity of the PDZ domain/ligand interaction. A conserved Gly-Leu-Gly-Phe (position 322-325 in PDZ3) motif and a positively charged residue (Arg318 in PSD-95 PDZ3) of PDZ domains mediate binding to the C-terminal carboxylate group.

The PDZ1 and PDZ2 domains of PSD-95 interact with several proteins including the simultaneous binding of the NMDA-type of ionotropic glutamate receptors and the nitric oxide (NO) producing enzyme nNOS. NMDA receptors are the principal mediators of excitotoxicity, i.e. glutamate-mediated neurotoxicity, which is implicated in neurodegenerative diseases and acute brain injuries, and although antagonists of the NMDA receptor efficiently reduce excitotoxicity by preventing glutamate-mediated ion-flux, they also prevent physiological important processes. Thus NMDA receptor antagonists have failed in clinical trials for e.g. stroke due to low tolerance and lack of efficacy. Instead, specific inhibition of excitotoxicity can be obtained by perturbing the intracellular nNOS/PSD-95/NMDA receptor complex using PSD-95 inhibitors.

PSD-95 simultaneously binds the NMDA receptor, primarily GluN2A and GluN2B subunits, and nNOS via PDZ1 and PDZ2, respectively. Activation of the NMDA receptor causes influx of calcium ions, which activates nNOS thereby leading to NO generation. Thus, PSD-95 mediates a specific association between NMDA receptor activation and NO production, which can be detrimental for the cells if sustained for a longer period, and is a key facilitator of glutamate-mediated neurotoxicity. Inhibition of the ternary complex of nNOS/PSD-95/NMDA receptor interaction by targeting PSD-95 is known to prevent ischemic brain damage in mice, by impairing the functional link between calcium ion entry and NO production, while the physiological function, such as ion-flux and pro-survival signaling pathways of the NMDA receptor remains intact. WO 2010/004003 discloses a concept of inhibiting PSD-95 by dimeric peptide ligands linked by a polyethylene glycol linker (PEG). These dimers simultaneously bind to the PDZ1 and PDZ2 domains of PSD-95.

Dimeric ligands targeting PSD-95 are under pre-clinical evaluation as a treatment for chronic pain (Andreasen et al, Neuropharmacol, 2013, 67, 193-200; Bach et al, PNAS USA, 2012, 109, 3317-3322). However, therapeutic peptides are generally susceptible to removal from the blood and degradation by renal clearance and hepatic metabolism. Therefore there is a need for improving the pharmacokinetic properties and thus increase stability and half-life of the dimeric peptide ligands.

### SUMMARY OF THE INVENTION

In order to address the stated problem of providing improved pharmacokinetic properties and increased in vivo stability of dimeric peptide ligands of PSD-95, the present invention describes a new class of compounds wherein two peptide ligands are linked by a linker such as NPEG linker, wherein one or more fatty acids or fatty acid derivatives have been conjugated either directly to the NPEG linker or via a further linker.

The present inventors have therefore developed derivatives of dimeric PSD-95 ligands having improved in vitro plasma half-lives compared to compounds without fatty acids attached, e.g. the compounds disclosed in WO2010/004003. Furthermore, the compounds show increased residence time in a subcutaneous depot upon subcutaneous administration.

In one aspect the present invention concerns a compound comprising a first peptide (P₁) and a second peptide (P₂), wherein P₁ and P₂ individually comprise at least two proteinogenic or non-proteinogenic amino acid residues, and wherein both P₁ and P₂ are conjugated to a first linker L₁ via their respective N-termini, and wherein L₁ comprises polyethylene glycol (PEG) wherein at least one oxygen atom of said PEG is substituted with a nitrogen atom to give NPEG, and wherein an albumin binding moiety is linked to the nitrogen atom of the NPEG by an amide bond, or via an optional linker L₂.

It has been demonstrated that the compounds of the present invention bind to PDZ1-2 of PSD-95 when electing P₁ and P₂ as defined herein. As PSD-95 is an important target for therapeutics, the present invention in one aspect concerns the compound as defined herein for use as a medicament.

More specifically, the compounds of the invention may in one aspect be used for the treatment or prophylaxis of an excitotoxic-related disease, or for prophylaxis and/or treatment of pain.

The compounds of the present invention can schematically be synthesized by a method comprising the steps of:

a) preparing a Ns-NPEG diacid linker,
b) preparing a peptide using Fmoc-based solid-phase peptide synthesis,
c) dimerizing Fmoc-deprotected peptide with Ns-NPEG diacid linker
d) coupling a fatty acid to the linker-dimer conjugate, optionally via an intermediate linker, such as an amino acid linker (L₂).

### DESCRIPTION OF THE DRAWINGS

FIG. 1: Structure of reference ligands, UCCB01-125 and UCCB01-144. Capital letters indicate L-amino acids, except for ‘N’ (nitrogen), ‘O’ (oxygen).

FIG. 2: Affinity for HSA of FA-linked dimeric ligands (1-12) and UCCB01-125 and UCCB01-144. Data shown as mean±SEM, n=3.

FIG. 3: Affinity to PSD-95 PDZ1-2 of FA-linked dimeric ligands (1-12) and UCCB01-125 and UCCB01-144 as determined by FP. A) Measured in TBS; B) Measure in TBS+HSA; C) Measured in TBS+HSA corrected for fu. Data shown as mean±SEM, n≧3.

FIG. 4: In vitro plasma stability of compounds (UCCB01-125, 1, 4, 7, 13). Calculated half-lives are: UCCB01-125: 1.7 h; 1: 23.6 h; 4, 7, and 13: >24 h.

FIG. 5: Plasma profiles of dimeric ligands UCCB01-125 (two doses) and compound 1, 4, 7 and 13 after s.c. administration in rats.

FIG. 6: Synthesis of FA-linked dimeric ligands (1-12). The reaction conditions of the synthesis illustrated in scheme 1 of this figure was as follows: (a) Fmoc-GABA-OH/Fmoc-(L)-Glu-OtBu/Fmoc-5-Ava, HATU, collidine, DMF (1 h×2), then 20% piperidine in DMF; (b) FA1/FA2/FA3/FA4, HBTU, DIPEA, DMF/DCM, 45 min, then TFA/TIPS/H₂O (90/5/5); (c) 0.5M LiOH, H₂O/ACN (75/25), 30 min, then TFA to pH<2. Triangle indicates that E and T are side-chain protected (tert-butyl).

FIG. 7: Mono saponification of octadecandioate dimethyl ester. Reaction conditions: (a) NaOH (1 eq.), MeOH, 45° C., O/N.

### DETAILED DESCRIPTION OF THE INVENTION

### I. Definitions

Amide bond: The term ‘amide bond’ as used herein is a chemical bond formed by a reaction between a carboxylic acid and an amine (and concomitant elimination of water). Where the reaction is between two amino acid residues, the bond formed as a result of the reaction is known as a peptide linkage (peptide bond);

Comprising: The term ‘comprising’ as used herein should be understood in an inclusive manner. Hence, by way of example, a composition comprising compound X, may comprise compound X and optionally additional compounds.

Dimer: The term dimer as used herein refers to two identical or non-identical chemical moieties associated by chemical or physical interaction. By way of example, the dimer can be a homodimer such as two identical peptides linked by a linker. The dimer may also be a heterodimer such as two different peptides linked by a linker. An example of a dimer is the PSD-95 inhibitor of the present invention which is a compound comprising two peptide or peptide analogues, that are covalently linked by means of a linker, wherein the peptides or peptide analogues are capable of binding to, or interacting with, PDZ1 and PDZ2 of PSD-95 simultaneously.

Dipeptide: The term ‘dipeptide’ as used herein refers to two natural or non-natural amino acids linked by a peptide bond.

Ethylene glycol moiety: The term ‘ethylene glycol moiety’ as used herein refers to the structural unit that constitutes a PEG or NPEG linker. Another name of an ‘ethylene glycol moiety’ is ‘oxyethylene’, and the chemical formula of the monomer unit is:

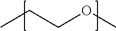

Fatty acid: The term fatty acid (abbreviated FA) as used herein typically refers to a carboxylic acid with a long aliphatic carbon chain, which can be either saturated or unsaturated. The fatty acid can be selected from Short-chain fatty acids (SOFA), Medium-chain fatty acids (MCFA), Long-chain fatty acids (LCFA) and Very long chain fatty acids (VLCFA). Short-chain fatty acids (SOFA) are fatty acids with aliphatic tails of fewer than six carbons (i.e. butyric acid). Medium-chain fatty acids (MCFA) are fatty acids with aliphatic tails of 6-12 carbons, which can form medium-chain triglycerides. Long-chain fatty acids (LCFA) are fatty acids with aliphatic tails 13 to 21 carbons. Very long chain fatty acids (VLCFA) are fatty acids with aliphatic tails longer than 22 carbons. The fatty acid of the present invention can be any suitable fatty acid or fatty acid derivative known by those of skill in the art.

Linker: The term ‘linker’ as used herein refers to one or more atoms forming a connection from one chemical entity to another. By way of example, the ‘first linker’ referred to herein is a PEG or NPEG, which joins the two PDZ-domain binding peptides by forming a link to each of their N-termini.

Non-proteinogenic amino acids: Non-proteinogenic amino acids also referred to as non-coded, non-standard or non-natural amino acids are amino acids which are not encoded by the genetic code. A non-exhaustive list of non-proteinogenic amino acids include α-amino-n-butyric acid, norvaline, norleucine, isoleucine, alloisoleucine, tert-leucine, α-amino-n-heptanoic acid, pipecolic acid, α,β-diaminopropionic acid, α,γ-diaminobutyric acid, ornithine, allothreonine, homocysteine, homoserine, β-alanine, β-amino-n-butyric acid, β-aminoisobutyric acid, γ-aminobutyric acid, α-aminoisobutyric acid, isovaline, sarcosine, N-ethyl glycine, N-propyl glycine, N-isopropyl glycine, N-methyl alanine, N-ethyl alanine, N-methyl β-alanine, N-ethyl β-alanine, isoserine and α-hydroxy-γ-aminobutyric acid.

NPEG: The term NPEG as used herein is a linker derivative of a PEG linker, but where one or more of the backbone oxygen atoms is replaced with a nitrogen atom

Ns-NPEG diacid linker: The ‘Ns-NPEG diacid linker’ is the structure where an NPEG linker is protected on the nitrogen with an ortho-nitrobenzenesulfonyl (Ns) protection group on the linker nitrogen, and where the termini of the NPEG linker comprise carboxylic acids. This chemical reagent or building block is used to dimerize the two peptide moieties, P₁ and P₂.

PDZ: The term ‘PDZ’ as used herein refers to Postsynaptic density protein-95 (PSD-95), Drosophila homologue discs large tumor suppressor (DIgA), Zonula occludens-1 protein (zo-1).

PEG: The term ‘PEG’ as used herein refers to a polymer of the ethylene glycol moiety discussed herein above. PEG has the chemical formula C₂ₙ₊₂H₄ₙ₊₆Oₙ₊₂, and the repeating structure is:

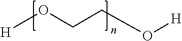

where for example 12 PEG moieties, or PEG12, corresponds to a polymer of 12 ethylene glycol moieties.

Pharmacokinetic profile: The term ‘pharmacokinetic profile’ as used herein refers to the in vivo characteristics of absorption into the blood stream, distribution into tissues, metabolization and excretion of the compounds described herein. An example of a parameter that is included in the pharmacokinetic profile is the in vitro half plasma half-life, which models the metabolization of the compounds by plasma proteases.

Proteinogenic amino acids: Proteinogenic amino acids, also referred to as natural amino acids include alanine, cysteine, selenocysteine, aspartic acid, glutamic acid, phenylalanine, glycine, histidine, isoleucine, lysine, leucine, methionine, asparagine, proline, pyrrolysine, glutamine, arginine, serine, threonine, valine, tryptophan and tyrosine.

PSD-95: The term ‘PSD-95’ as used herein refers to postsynaptic density protein-95.

PSD-95 inhibitor: The term ‘PSD-95 inhibitor’ as used herein refers to a compound that binds to PDZ1, PDZ2, or both PDZ1 and PDZ2 of PSD-95 and inhibits the protein-protein interactions facilitated by these PDZ domains in a cell. An example of an interaction that is inhibited by a PSD-95 inhibitor is the ternary complex formation between nNOS, PSD-95 and the NMDA receptor.

### II. Dimeric Compounds with Improved Plasma Half-Life

Dimeric ligands targeting PSD-95 are under pre-clinical evaluation as a treatment for chronic pain and ischemic stroke (Andreasen et al, Neuropharmacol, 2013, 67, 193-200; Bach et al, PNAS USA, 2012, 109, 3317-3322). However, therapeutic peptides in general are susceptible to degradation by proteases and elimination by renal filtration and/or hepatic metabolism (Tang et al, J Pharm Sci, 2004, 93, 2184-204). Due to the limited size of the dimeric ligands, they are likely to be cleared from the blood by renal filtration, since the kidneys generally filter out compounds with a molecular weight below 60 kDa (Dennis et al, J Biol Chem 2002, 277, 35035-43). To make dimeric peptide ligands more suitable for clinical utility in a chronic setting such as neuropathic pain, the dosing regimen should be as simple as possible, preferably once-daily, to increase compliance (Claxton et al, Clin Ther 2001, 23, 1296-310). Furthermore, self-administration by the patient via appropriate routes, such as subcutaneous (s.c.) administration, is preferred over i.v. (intravenous) injection. The main limitation to s.c. administration is the requirement for a low injection volume (Dychter et al, J Infus Nurs 2012, 35, 154-60) requiring the drug to be highly potent, highly concentrated, soluble, and degraded and excreted slowly from the circulation. Furthermore, the drug should be stable in the injection depot and absorbed slowly into the circulation to protract the action of the drug (Havelund et al, Pharm Res 2004, 21, 1498-504). Thus, the pharmacokinetic profile and in vivo half-life of the dimeric peptide ligands should be further optimized to account for these issues.

### Albumin Binding

Human Serum Albumin (HSA), which is the most abundant protein in human serum with 55% of the total serum protein (Elsadek et al, J Control Release 2012, 157, 4-28), offers an opportunity to solve these problems. The average blood concentration of HSA is 520-830 μM, and the molecular weight is approximately 66.5 kDa (Kragh-Hansen et al, Biol Pharm Bull 2002, 25, 695-704). HSA is abundant in blood, muscular tissue and skin (Sleep et al, Biochim Biophys Acta 2013, 1830, 5526-34), but not present inside neurons under normal conditions. It may however enter neurons in disease states where the brain-blood barrier (BBB) is compromised, such as stroke (Løberg, APMIS 1993, 101, 777-83). HSA serves as a transport and depot protein for numerous endogenous ligands such as fatty acids (FAs), hemin, bilirubin and tryptophan (Simard et al, PNAS USA, 2005, 102, 17958-63; Kragh-Hansen et al, Biol Pharm Bull 2002, 25, 695-704) and drugs such as warfarin and diazepam as well as metal ions (Yamasaki et al, Biochim Biophys Acta 2013, 1830, 5435-43).

The structure of HSA has been studied extensively, and more than 90 different x-ray structures of HSA are deposited in the Protein Data Bank (PDB, accessed 26/09/2013) with 71 different ligands. HSA is a heart-shaped molecule with approximate dimensions of 80×80×30 Å and consists of three similar domains (I, II and III), that are further divided into two subdomains (a and b) (Sugio et al, Protein Eng 1999, 12, 439-46). The majority of drugs bind in Sudlow's Site I (also called the Warfarin site) and II (also called the Diazepam site) (Elsadek et al, J Control Release 2012, 157, 4-28; Yamasaki et al, Biochim Biophys Acta 2013, 1830, 5435-43), named after the pioneering work of Sudlow and colleagues who in 1975 identified the sites by fluorescence spectroscopy (Sudlow et al., Mol Pharmacol 1975, 11, 824-32). The two drug binding sites have since been mapped and are found within subdomain IIa (with contribution from residues in subdomains IIIa and IIb) and IIIa, respectively (Yamasaki et al, Biochim Biophys Acta 2013, 1830, 5435-43). One important exception to the general binding of ligands in Sudlow's site I and II are the fatty acids (FAs).

The concept that high HSA binding of a drug increases the half-life of the drug has been known since the 1970's. However, the specific concept of using HSA to increase the half-life of therapeutic peptides and proteins has evolved more slowly, with a doubling in the number of yearly publications from 2002 (˜250 publications/year) to 2010 (˜500 publications/year) (Elsadek et al, J Control Release 2012, 157, 4-28). Several peptide-based HSA binding moieties have been described, including HSA-binding sequences identified by phage-display (Dennis et al, J Biol Chem 2002, 277, 35035-43), isolated from natural sources (Jonsson et al, Protein Eng Des Sel 2008, 21, 515-27 and so-called adnectins (Lipovsek et al, Protein Eng Des Sel 2011, 24, 3-9). These may however be susceptible to protease degradation, which may be particularly a problem in the current case of s.c. administration, where the developed compounds are supposed to reside in the s.c. depot for several hours.

### Overall Structure

In order to improve the pharmacokinetic profile the present inventors investigate the influence of fatty acids and linker types on HSA affinity, affinity for PSD-95 and hydrophobicity of the generated compounds. In doing so, novel compounds have been identified that provide the desired HSA-binding profile and enhanced stability in human plasma.

Thus in one aspect the present invention concerns a compound comprising a first peptide (P₁) and a second peptide (P₂), wherein P₁ and P₂ individually comprise at least two proteinogenic or non-proteinogenic amino acid residues, and wherein both P₁ and P₂ are conjugated to a first linker L₁ via their N-termini, and wherein L₁ comprises polyethylene glycol (PEG) wherein at least one oxygen atom of said PEG is substituted with a nitrogen atom to give NPEG, and wherein an albumin binding moiety is linked to the nitrogen atom of the NPEG by an amide bond, or via an optional linker L₂.

In certain embodiments said compound are of the general formula (I):

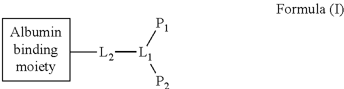

While the albumin binding moiety can be any suitable chemical group binding albumin, it is preferred that the albumin binding moiety is a fatty acid (FA). In one embodiment the compound thus has the general formula (II):

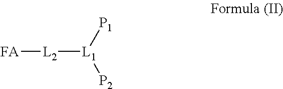

The fatty acid can be any suitable fatty acid such as a saturated or an unsaturated fatty acid. As illustrated in formulas (I) and (II) above, the albumin binding moiety such as the fatty acid, may optionally be linked to the nitrogen atom of an NPEG linker (L₁) via a second linker L₂. In embodiments wherein the second linker L₂ is included, that linker comprises a nitrogen atom.

In one embodiment the compound according to the present invention has the generic structure of formula (III) or (IV):

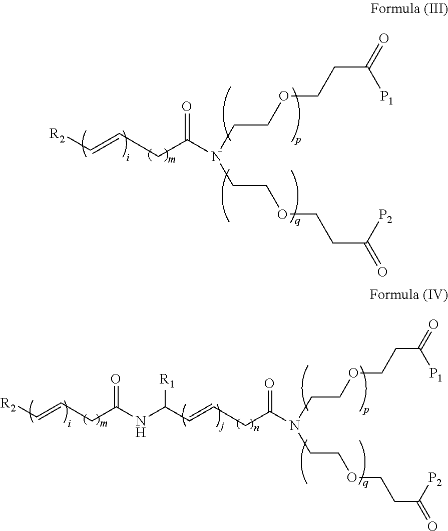

wherein
R₁ and R₂ individually are selected from the group consisting of H and COOH,
n is an integer 0 to 48,
m is an integer 1 to 48,
p is an integer 0 to 28,
q is an integer 0 to 28,
i is an integer 0 to 12,
j is an integer 0 to 12
and wherein P₁ and P₂ are individually selected from peptides comprising at least two proteinogenic or non-proteinogenic amino acid residues.

### The First Linker (L₁)

The properties exhibited by the fatty acid on the active peptides P₁ and P₂ are dependent on the manner in which these moieties are linked. The linking is achieved via the first linker L₁ and the second linker L₂. The first linker L₁, which to some extent has been described in WO 2012/156308, consists of a number of ethylene glycol moieties forming a polyethylene glycol, wherein one of the oxygen atoms has been replaced by a nitrogen atom to form an NPEG linker. The NPEG linker can be illustrated as a nitrogen atom flanked on each side by a number (p, q) of ethylene glycol moieties.

The number of ethylene glycol moieties flanking the nitrogen atom can be varied in different embodiments of the present invention. In one embodiment the number of ethylene glycol moieties (p) on one side is equal to the number of ethylene glycol moieties on the opposite side, i.e. p=q. In other embodiments p>q or p<q.

In one embodiment the sum of p and q is an integer between 0 and 28, such as wherein the number of ethylene glycol moieties, p is selected from 0, 1, 2, 3, 4, 5, 6, 7, 8, 9, 10, 11, 12, 13, 14, 15, 16, 17, 18, 19, 20, 21, 22, 23, 24, 25, 26, 27 or 28.

In one embodiment the number of ethylene glycol moieties, p is 1 to 4, as that range of p provides the highest affinity towards PSD-95.

In one embodiment the number of ethylene glycol moieties, q is selected from 0, 1, 2, 3, 4, 5, 6, 7, 8, 9, 10, 11, 12, 13, 14, 15, 16, 17, 18, 19, 20, 21, 22, 23, 24, 25, 26, 27 or 28 ethylene glycol moieties.

In one embodiment the number of ethylene glycol moieties, q is 1 to 4, as that range of q provides the highest affinity towards PSD-95.

In one embodiment the total number of ethylene glycol moieties p+q is between 2 and 12, as linker length within that range provides the highest affinity towards PSD-95.

In one embodiment the number of ethylene glycol moieties, p+q is 4, as linker length of that range provides the very highest affinity towards PSD-95.

In another embodiment the number of ethylene glycol moieties, p+q is 6, as linker length of that range provides a very high affinity towards PSD-95.

### The Second Linker (L₂)

The second linker L₂ is optional and can be included or excluded depending on the physical or chemical properties required for a particular purpose. When present, the second linker L₂ comprises a nitrogen atom. The second linker L₂ can e.g. be selected from the group consisting of γ-Glu, γ-butyric acid (GABA), 5-amino valeric acid (5-Ava), proteinogenic amino acids, non-proteinogenic amino acids, and any compound having the general formula H₂N-[Q]-COOH, wherein Q is any suitable atom or atoms or molecule.

As mentioned herein above, the second linker may comprise a repetitive carbon moiety thus forming e.g. an alkyl or an alkenyl chain. The number of repetitive units (i) and/or (m) can be varied depending on the desired properties. Thus i and/or m are integers which individually can be selected from the group consisting of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 and 48.

In one embodiment n is an integer between 1 and 3 such as in an embodiment where n=1 or n=2 or n=3.

In certain embodiments i and/or j is an integer between 0 and 12, e.g. an integer selected from the group consisting of 0, 1, 2, 3, 4, 5, 6, 7, 8, 9, 10, 11 and 12.

In certain embodiments the linkers L₁ and L₂ have been elected so that the compound according to the invention is selected from the group consisting of:

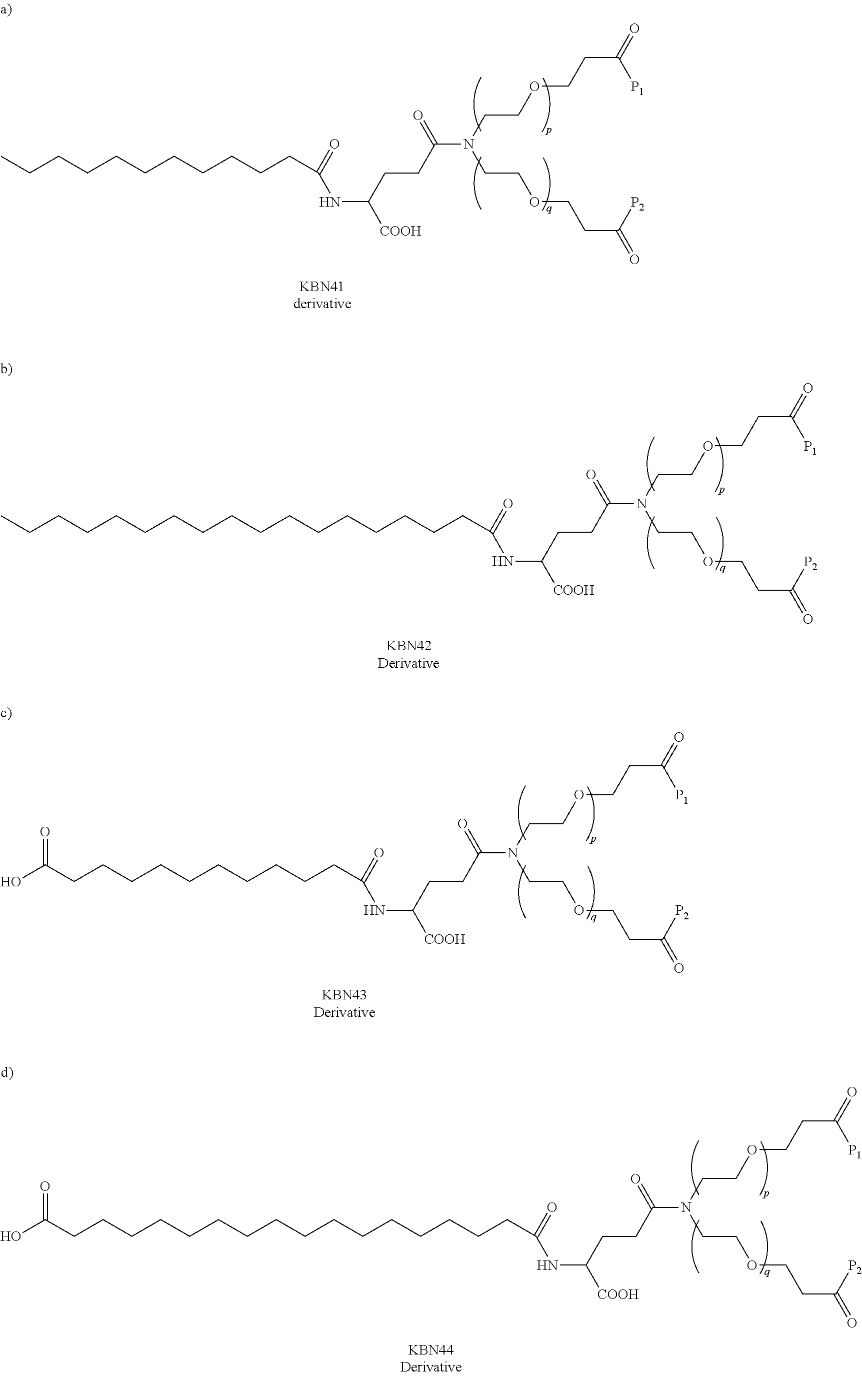

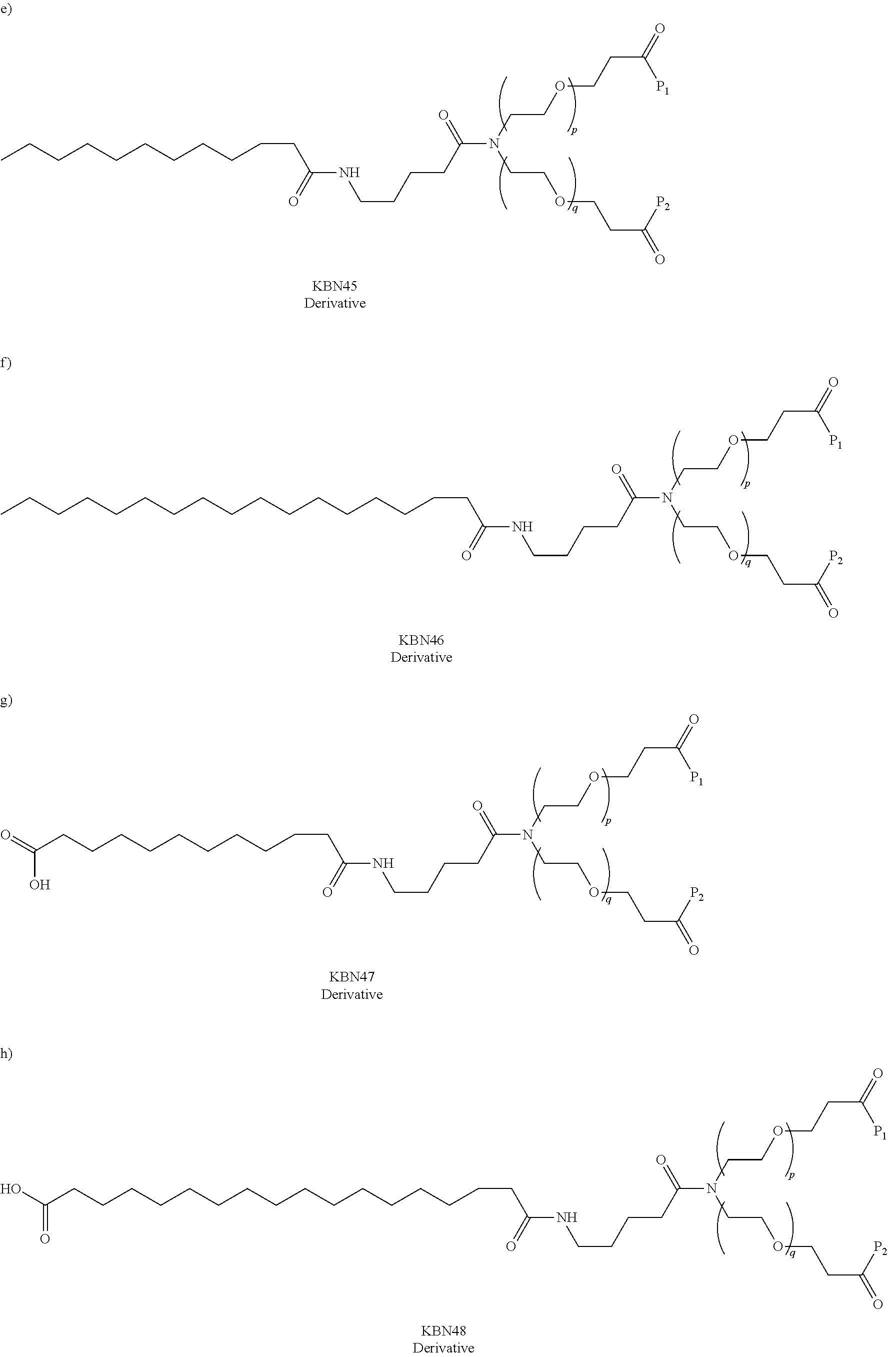

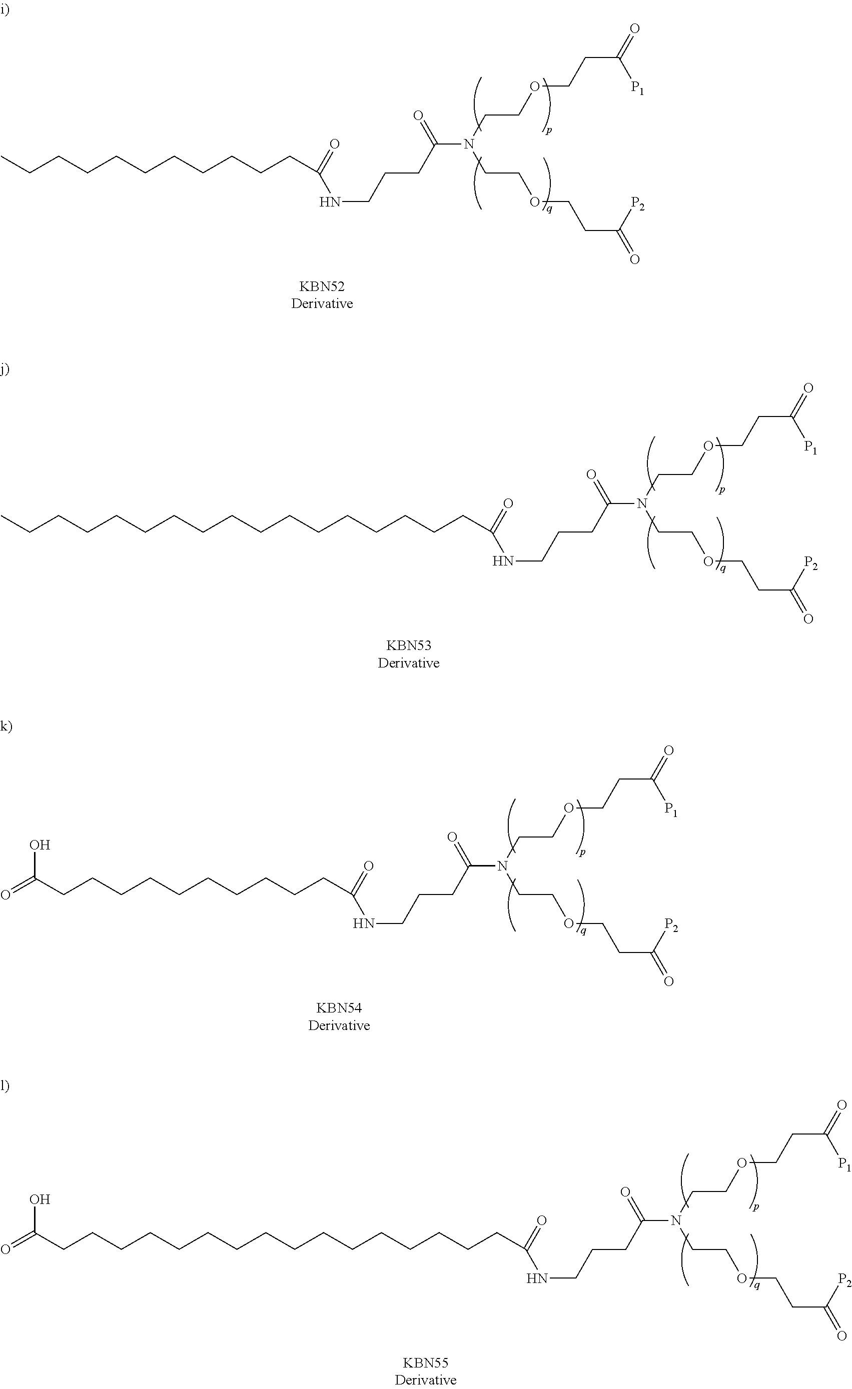

In a further embodiment the linkers have been elected so that the compound of the invention is selected from the group consisting of:

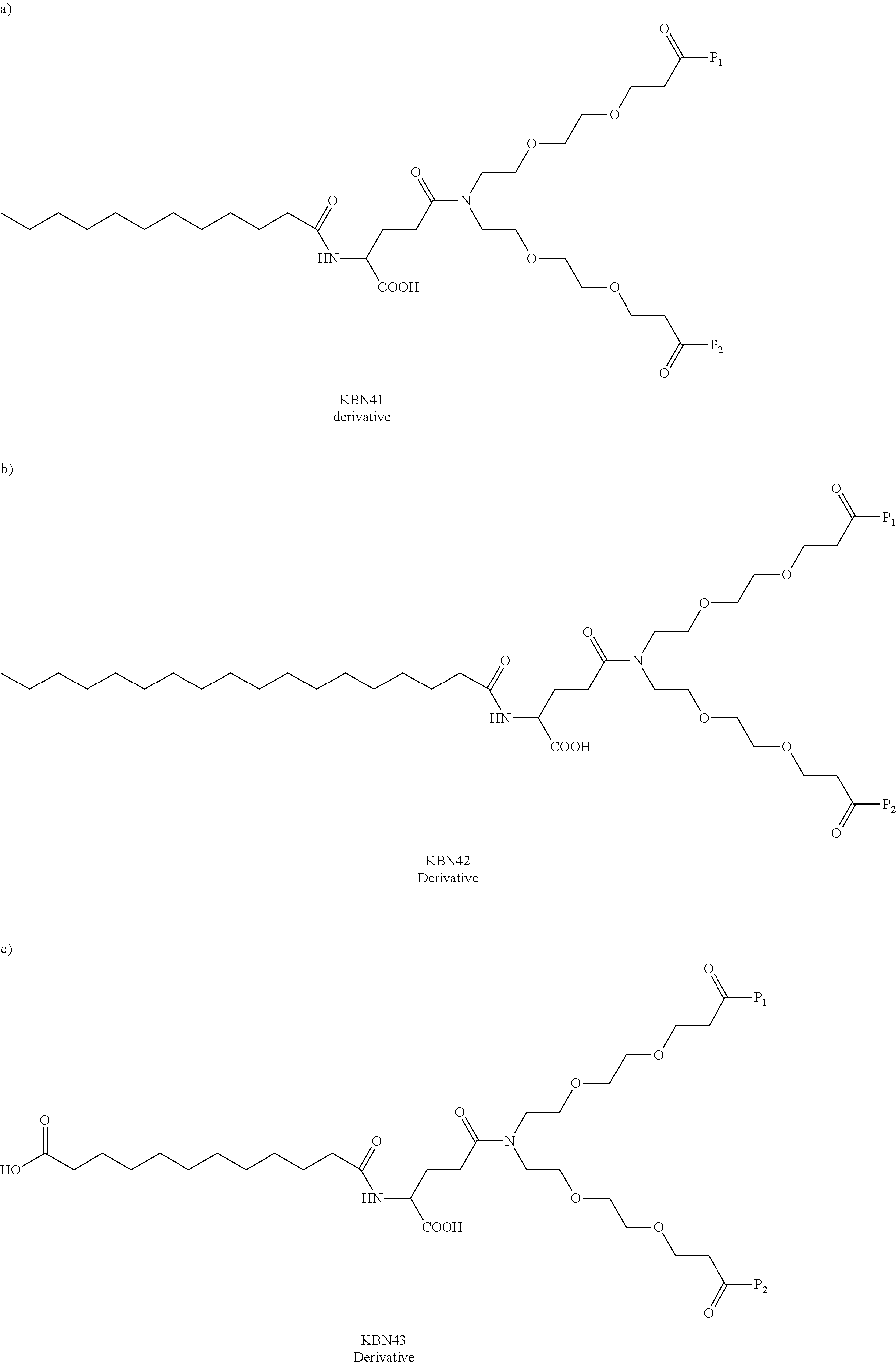

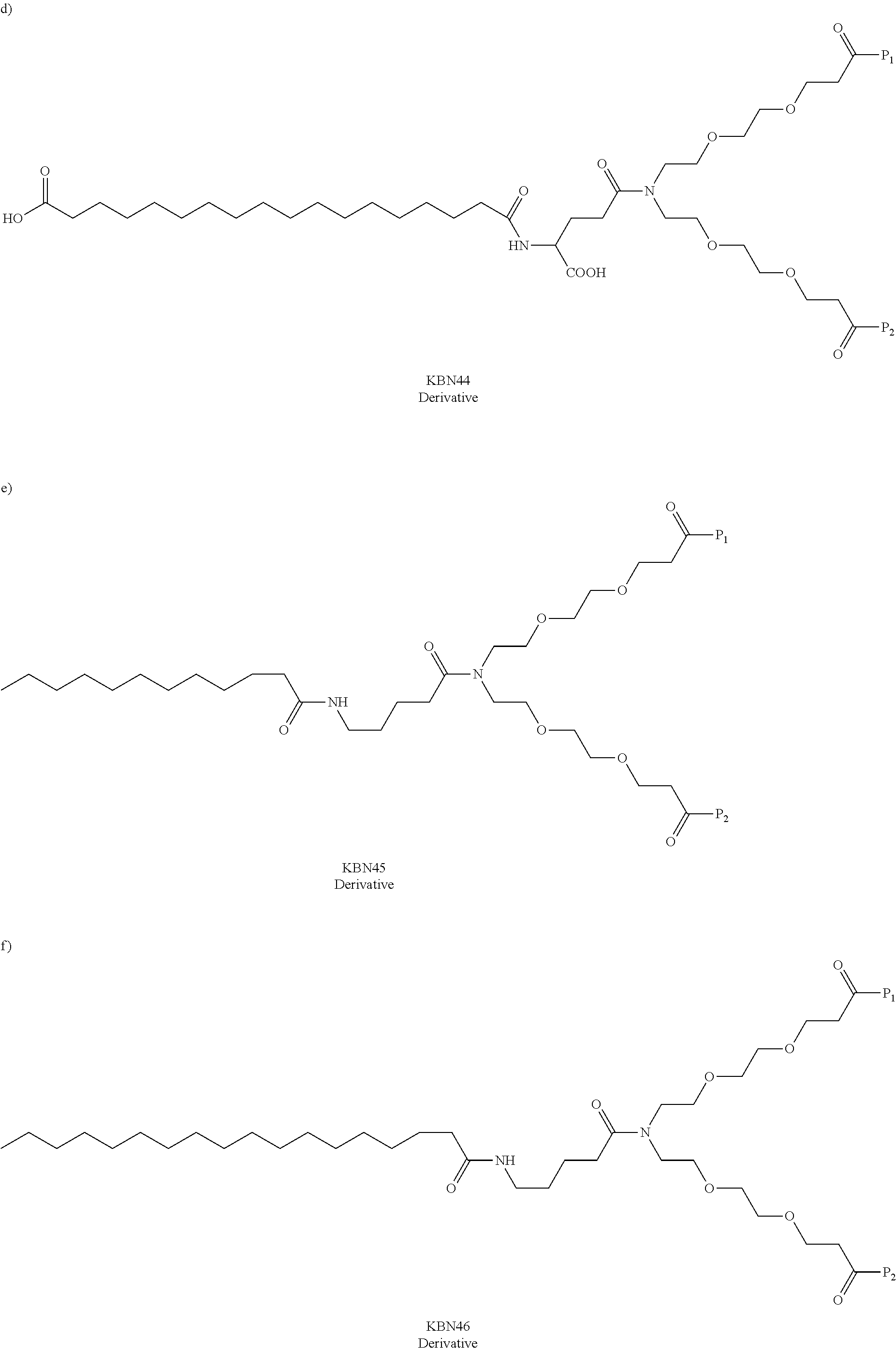

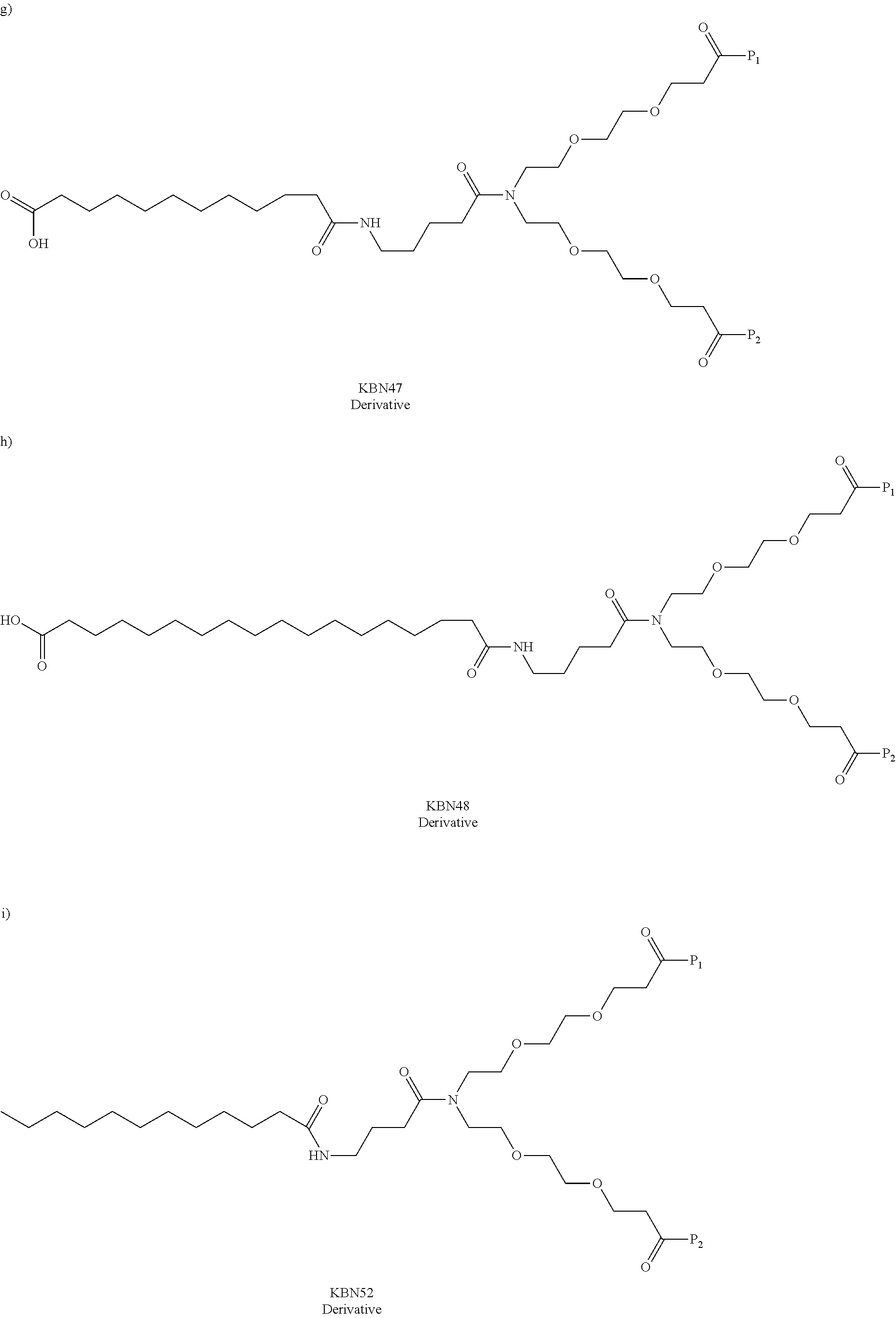

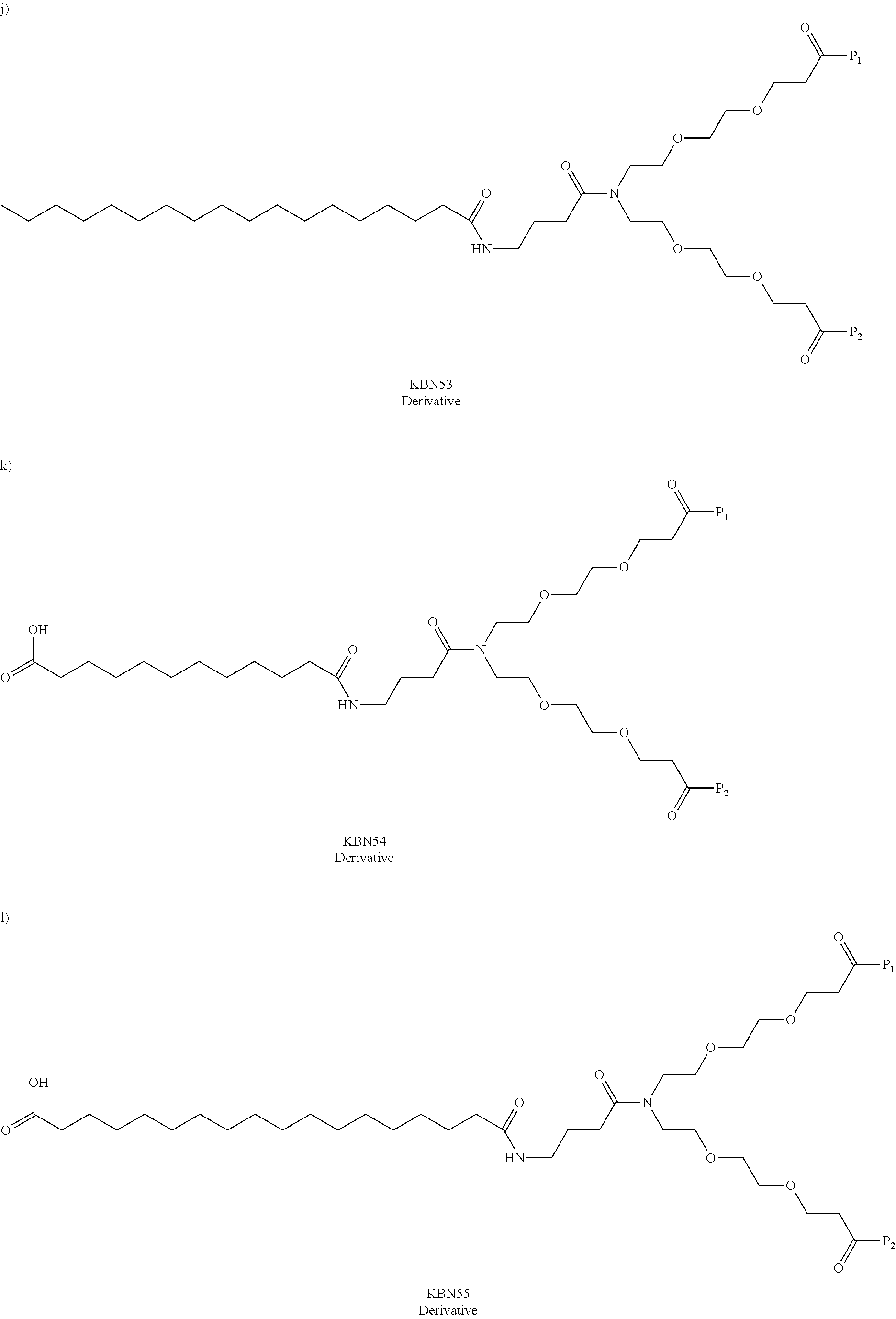

### Fatty Acid (FA)

A fatty acid is a carboxylic acid with an aliphatic tail (chain), which is either saturated or unsaturated. Most naturally occurring fatty acids have a chain of an even number of carbon atoms, from 4 to 28. Fatty acids are usually derived from triglycerides or phospholipids. When they are not attached to other molecules, they are known as free fatty acids.

Fatty acids that have carbon-carbon double bonds are known as unsaturated fatty acids while fatty acids without double bonds are known as saturated. Unsaturated fatty acids have one or more double bonds between carbon atoms. In certain embodiments the compound of the present invention comprises an unsaturated fatty acid. In such embodiments the indicator (i) and/or (j) of generic formulas (III), (IV), (V) or (VI) is an integer individually selected from an integer between 0 and 12, such as an integer selected from the group consisting of 0, 1, 2, 3, 4, 5, 6, 7, 8, 9, 10, 11 and 12.

Unsaturated fatty acids are available in either cis or trans configuration, or in a mixture of both.

A cis configuration means that adjacent hydrogen atoms are on the same side of the double bond. The rigidity of the double bond freezes its conformation and, in the case of the cis isomer, causes the chain to bend and restricts the conformational freedom of the fatty acid. The more double bonds the chain has in the cis configuration, the less flexibility it has. When a chain has many double bonds with cis configuration, it becomes quite curved in its most accessible conformations. For example, oleic acid, with one double bond, has a “kink” in it, whereas linoleic acid, with two double bonds, has a more pronounced bend. α-Linolenic acid, with three double bonds, favors a hooked shape. The effect of this is that, in restricted environments, such as when fatty acids are part of a phospholipid in a lipid bilayer, or triglycerides in lipid droplets, cis double bonds limit the ability of fatty acids to be closely packed, and therefore could affect the melting temperature of the membrane or of the fat.

A trans configuration, by contrast, means that the next two hydrogen atoms are bound to opposite sides of the double bond. As a result, they do not cause the chain to bend much, and their shape is similar to straight saturated fatty acids.

It is within the scope of the present invention to elect any fatty acid suitable for the intended purpose including cis, trans or mixed fatty acids.

The compound according to the present invention may comprise any suitable fatty acid or fatty acid derivative. In one embodiment the fatty acid is a fatty acid as defined in generic formulas (III) or (IV) wherein m is an integer selected from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 and 48 such as wherein m is an integer between 10 and 16, e.g. wherein m=10 or wherein m=16, which without doubt ascertain a high degree of HSA interaction (Table 1).

The fatty acid of the invention may be a C₄-C₂₂ fatty acid or a fatty acid selected from the group consisting of caprylic acid, capric acid, lauric acid, myristic acid, palmitic acid, stearic acid, arachidic acid, behenic acid, lignoceric acid, cerotic acid, myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid and docosahexaenoic acid.

Peptides (P₁ and P₂)

The compound of the present invention comprises two peptides P₁ and P₂.

P₁ and P₂ may individually be any peptide, each comprising at least two amino acid residues and the dimeric compound of the present invention may thus be adapted for the intended purpose.

In a preferred embodiment the compound of the present invention is a PSD-95 inhibitor wherein the two peptides bind to PDZ1-2 of PSD-95. Thus, in certain embodiments the compound is a compound as defined in any one of the generic formulas (I), (II), (III) or (IV), wherein:

P₁ comprises the amino acid sequence X₄X₃X₂X₁ (SEQ ID NO: 1), and

P₂ comprises the amino acid sequence Z₄Z₃Z₂Z₁ (SEQ ID NO: 2),
wherein
   a) X₁ and/or is an amino acid residue selected from I, L and V,
   b) X₂ and/or Z₂ is an amino acid residue selected from A, D, E, Q, N, S, V, N-Me-A, N-Me-D, N-Me-E, N-Me-Q, N-Me-N, N-Me-S and N-Me-V,
   c) X₃ and/or Z₃ is an amino acid residue selected from S and T,
   d) X₄ and/or Z₄ is an amino acid residue selected from E, Q, A, N and S,
wherein X₁ and Z₁ both individually represent the ultimate C-terminal amino acid residue comprising a free carboxylic acid.

Thus, in certain embodiments the compound according to the present invention has the generic structure of formula (V) or (VI):

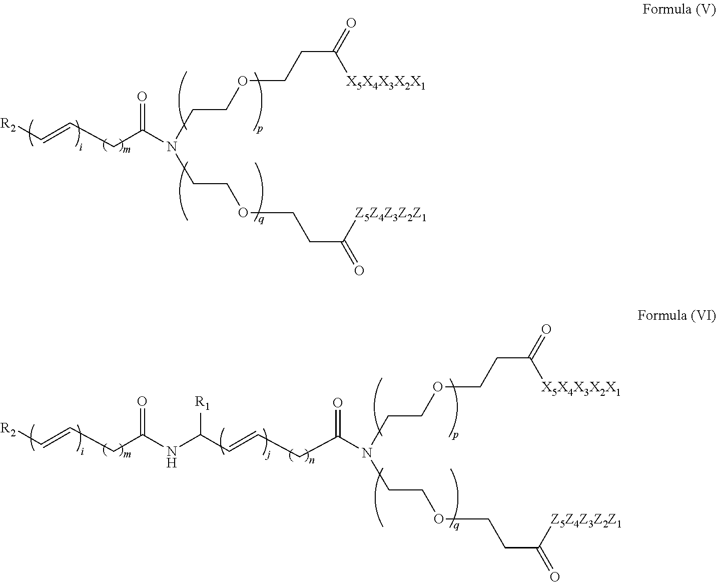

wherein
R₁ and R₂ individually are selected from the group consisting of H and COOH,
n is an integer 0 to 48,
m is an integer 1 to 48, and
p is an integer 0 to 28,
q is an integer 0 to 28,
i is an integer 0 to 12,
j is an integer 0 to 12 X₅ and/or Z₅ are/is an optional amino acid residue, a peptide or a polypeptide,
X₄ and/or Z₄ is an amino acid residue selected from E, Q, A, N and S,
X₃ and/or Z₃ is an amino acid residue selected from S and T,
X₂ and/or Z₂ is an amino acid residue selected from A, D, E, Q, N, S, V, N-Me-A, N-Me-D, N-Me-E, N-Me-Q, N-Me-N, N-Me-S and N-Me-V
X₁ and/or Z₁ is an amino acid residue selected from I, L and V.

If X₅ is a single amino acid residue it is selected from proteinogenic and non-proteinogenic amino acid residues.

In one embodiment X₅ is an amino acid residue selected from the group consisting of I, A, L and V.

X₅ may also be a peptide or polypeptide having an amino acid sequence consisting of between 2 to 100 amino acid residues, wherein the C terminus of said peptide or polypeptide is an amino acid residue selected from the group consisting of I, A, L and V.

In certain embodiments of the present invention X₅ is a peptide comprising 2 to 100 residues, such as 2 to 90 amino acid residues, such as 2 to 80 amino acid residues, such as 2 to 70 amino acid residues, such as 2 to 60 amino acid residues, such as 2 to 50 amino acid residues, such as 2 to 40 amino acid residues, such as 2 to 30 amino acid residues, such as 2 to 20 amino acid residues, such as 2 to 10 amino acid residues, such as 2 to 9 amino acid residues, such as 2 to 8 amino acid residues, such as 2 to 7 amino acid residues, such as 2 to 6 amino acid residues, such as 2 to 5 amino acid residues, such as 2 to 4 amino acid residues, such as 2 to 3 amino acid residues, wherein the C terminus is an amino acid selected from the group consisting of I, A, L and V

While the concept of the present invention is generally applicable as illustrated in generic formulas (I), (II), (III), (IV), (V) and (V), the present inventors have prepared a number of compounds within the present invention, comprising a peptide motif of P₁ and P₂ being suitable for binding to PDZ1-2 of PSD-95.

Thus in one embodiment, the compound according to the present invention is selected from the group consisting of:

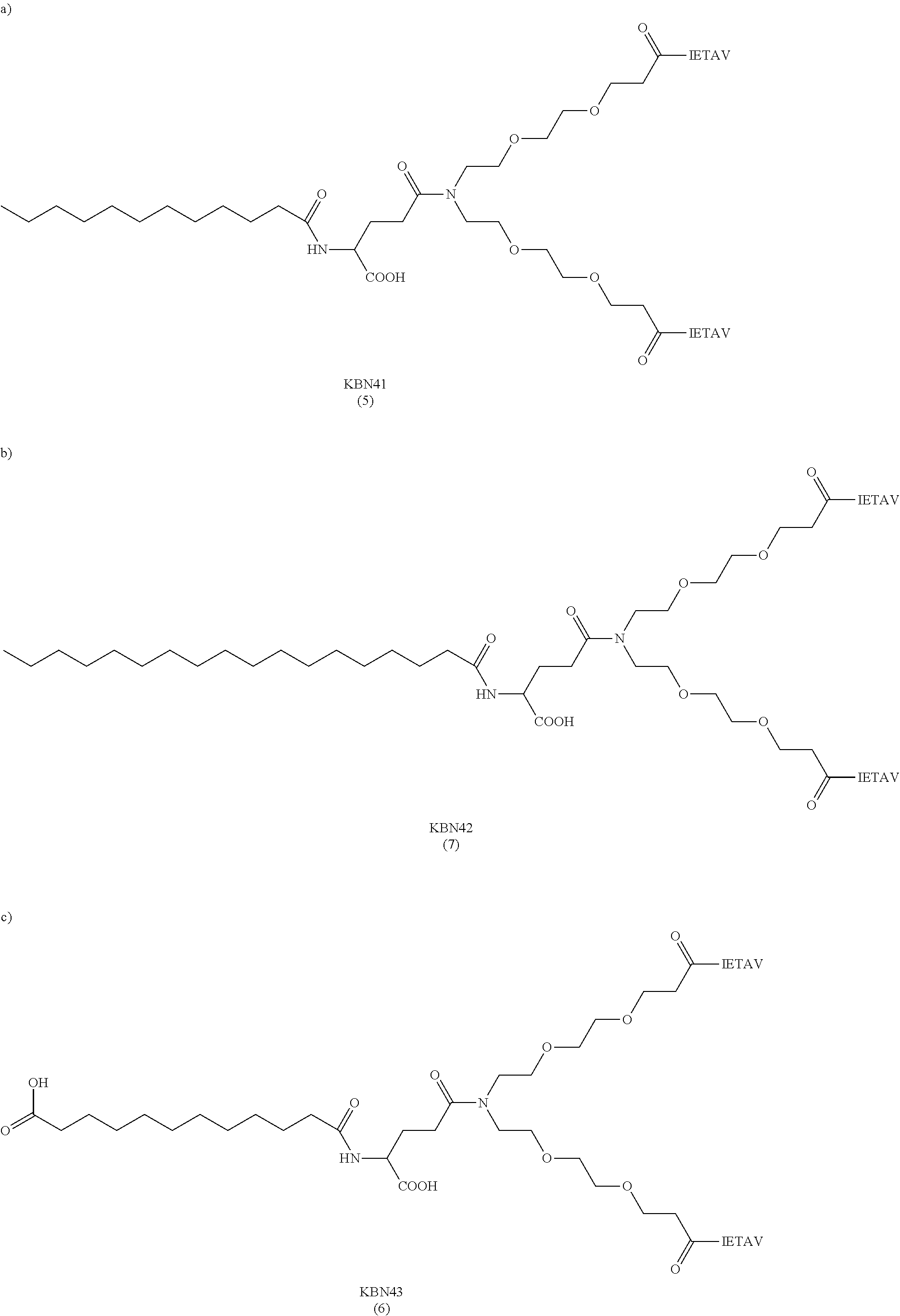

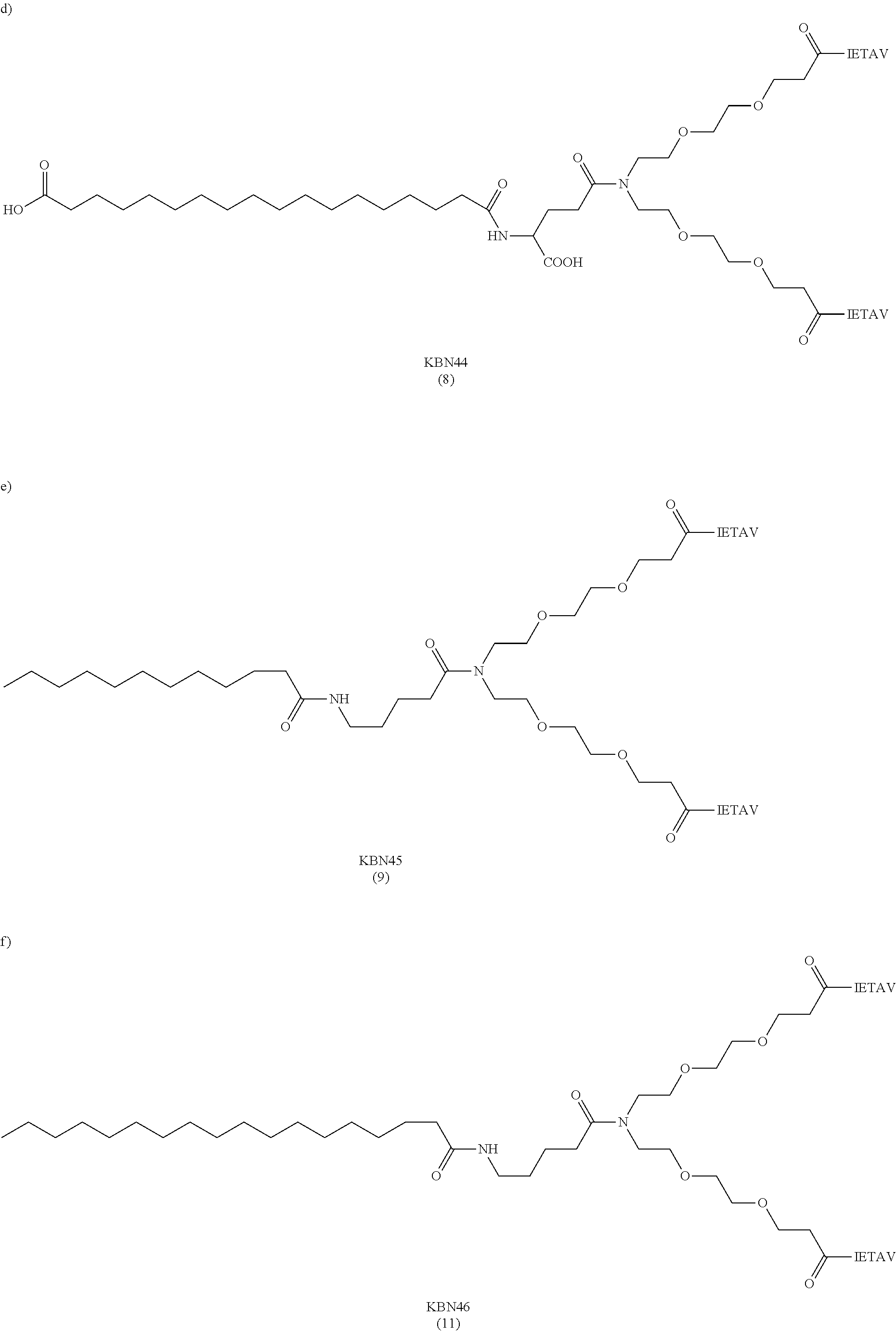

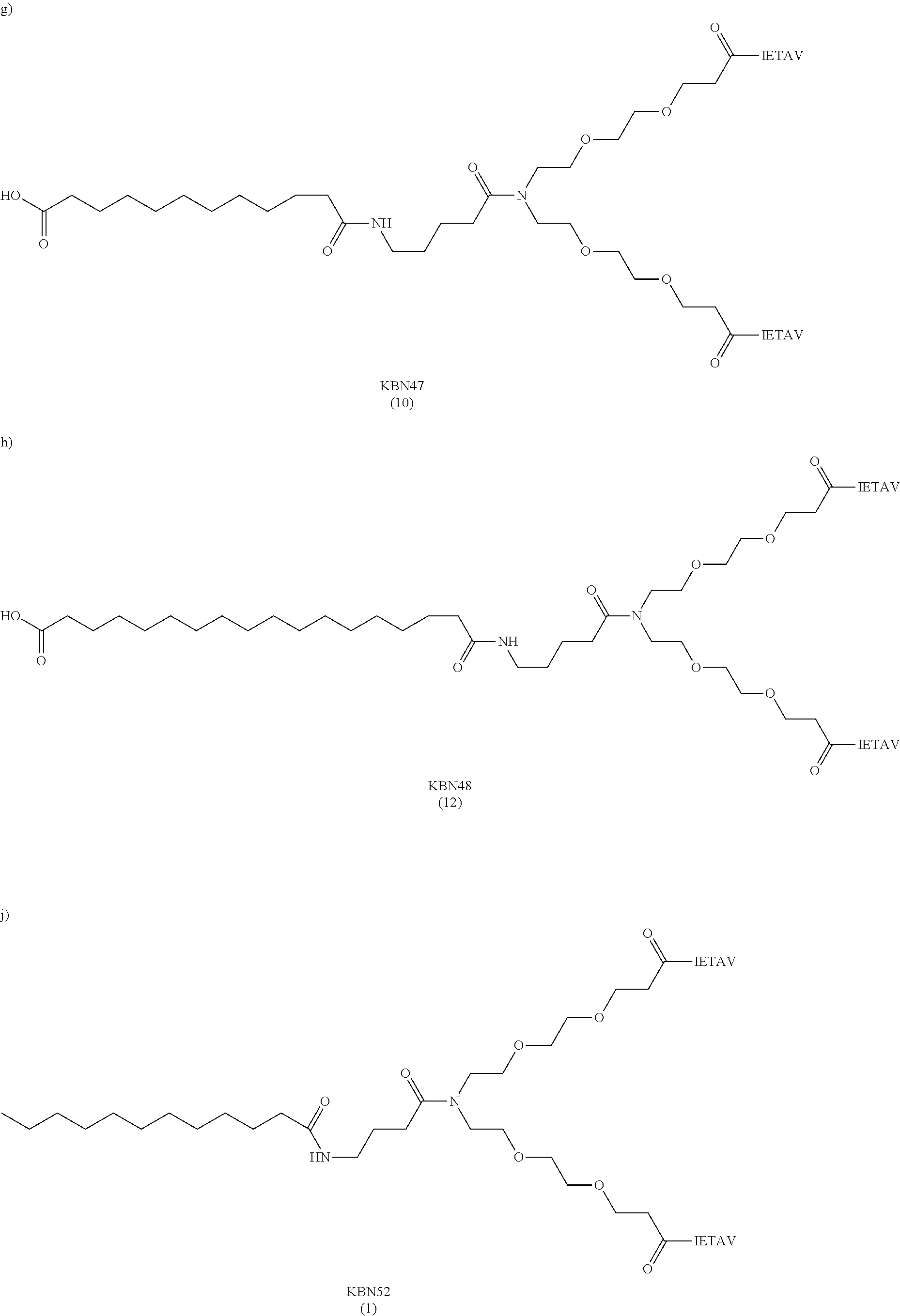

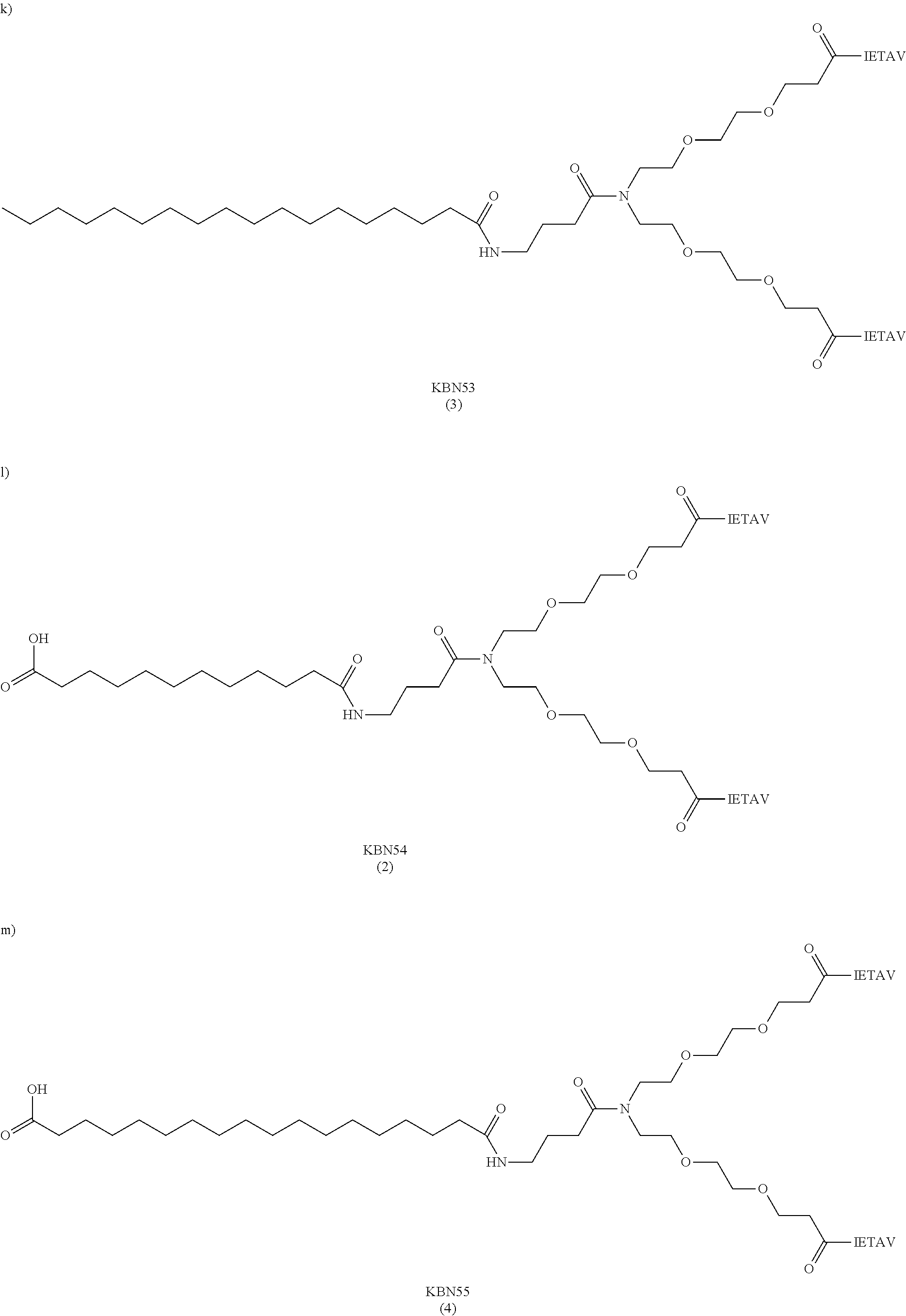

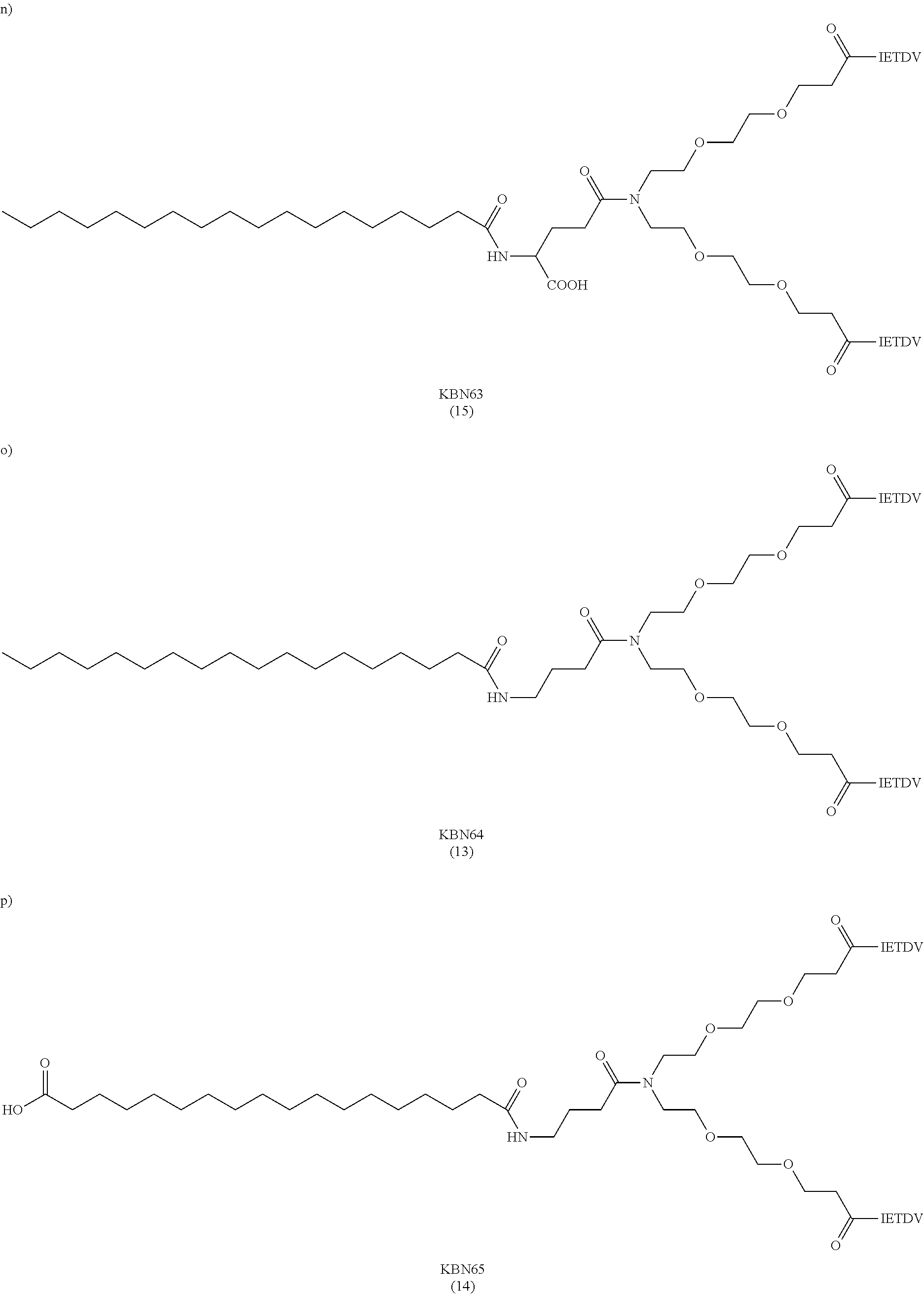

### Salt Forms

The compound as defined herein can be in the form of a pharmaceutically acceptable salt or prodrug of said compound. In one embodiment of the present invention the compound as defined in any one of the general formulas (I), (II), (III), (IV), (V) and (VI) can be formulated as a pharmaceutically acceptable addition salt or hydrate of said compound, such as but not limited to K⁺, Na⁺, as well as non-salt e.g. H.

### III. Medical Use

In one aspect the compound of the present invention as defined herein, is for use as a medicament.

In one embodiment the compound as defined herein is for use in the treatment or prophylaxis of pain.

In another embodiment the compound as defined herein is for use in the treatment or prophylaxis of an excitotoxic-related disease.

In a further embodiment the disease treatable by the compound of the present invention is ischemic or traumatic injury of the CNS.

### IV. Synthesis

The fatty acid derivatized PSD-95 inhibitors of the present invention as defined herein may be manufactured by a method comprising the general steps of:

a) preparing a Ns-NPEG diacid linker,
b) preparing a peptide using Fmoc-based solid-phase peptide synthesis,
c) dimerizing Fmoc-deprotected peptide with Ns-NPEG diacid linker
d) coupling a fatty acid to the linker-dimer conjugate, optionally via an intermediate linker, such as an amino acid linker (L₂).

The compounds of the present invention can in one embodiment be synthesized as defined in the following.

### Ns-NPEG Diacid Linker:

The ortho-nitrobenzenesulfonyl (Ns)-protected NPEG linker is produced either on solid-phase or in solution.

The solid-phase procedure typically starts by loading a solid support useful for solid-phase peptide synthesis, such as 2-chlorotrityl chloride resin, with Fmoc-NH-PEG-CH₂CH₂COOH, using appropriate organic solvent for the specific resin (e.g. DCM, DMF, ACN, THF) and a base (e.g. DIPEA, DBU, collidine, NMM)

The Fmoc group can be removed by base (e.g. piperidine, dimethylamine, morpholine, piperazine, dicyclohexylamine, DMAP) in appropriate solvent (e.g. DMF, DCM, ACN, THF).

Ortho-nitrobenzenesulfonyl chloride can be coupled to the free amine using base (e.g. DIPEA, DBU, collidine, NMM) and appropriate solvent (e.g. THF, DCM) to get Ns-NH-PEG-CH₂CH₂COO-Resin.

The second part of the linker product can be connected to the resin-bound linker-part by the use of Mitsunobu-chemistry. Resin is treated with triphenylphosphine, HO-PEG-CH₂CH₂COOtBu, solvent, and ester- or amide reagents of azodicarboxylic acid (e.g. diisopropyl azodicarboxylate, DIAD; diethyl azodicarboxylate, DEAD; 1,1′-(Azodicarbonyl)-dipiperidine, ADDP).

The final Ns-NPEG diacid linker is obtained by treating the resin with acid, such as trifluoroacetic acid (TFA).

The solution-phase procedure can be performed by protection of the amine group of NH₂—PEG-CH₂CH₂COOtBu with Ns, followed by Mitsunobu chemistry in solution using triphenylphosphine and DIAD, DEAD, or ADDP, or similar reagents, HO-PEG-CH₂CH₂COOtBu, and appropriate solvent (THF, DCM). Final Ns-protected NPEG-linker is then obtained by treatment with acids, such as TFA.

### Peptide Synthesis:

The peptide sequence is synthesized by Fmoc-based solid-phase peptide synthesis using a solid support, such as 2-chlorotrityl chloride resin or Wang resin, Fmoc-protected amino acids, base, coupling reagents (e.g. HBTU [N,N,N′,N′-Tetramethyl-O-(1H-benzotriazol-1-yl)uronium hexafluorophosphate], O-(7-azabenzotriazol-1-yl)-N,N,N′,N′-tetramethyluronium hexafluorophosphate [HATU], PyBOB, DIC/HOBt) and solvents. Alternatively to coupling reagents, activated ester of Fmoc-protected amino acids (e.g. pentafluorophenyl, succinimide) can be used.

### Dimerization:

The Fmoc-deprotected resin-bound peptide is dimerized with the Ns-NPEG diacid linker by an on-resin dimerization process by repetitive treatments of the resin with the Ns-NPEG diacid linker in sub-stoichiometric amounts (e.g. 1/6), base, coupling reagent, and appropriate solvents (e.g. DMF, DCM, THF). Alternatively to coupling reagents, activated ester of the Ns-NPEG linker can be used.

The dimerization process can also be formed in solution using either the activated ester (e.g. pentafluorophenyl, succinimide) of the Ns-NPEG linker together with 1-Hydroxy-7-azabenzotriazole (HOAt) or Hydroxybenzotriazole (HOBt) and appropriate side chain-protected peptide (e.g. tert-butyl) in solvent (e.g. ACN, DMF, DCM, THF). Also, dimerization in solution can be performed using the Ns-NPEG diacid linker, coupling reagents (e.g. HBTU, HATU etc), base and solvents.

The Ns-group is removed by mercaptoethanol and 1,8-diazabicyclo[5.4.0]undec-7-ene (DBU) or by sodium thiophenolate.

### Linker and Fatty Acid Conjugation:

The amino acid linker can be coupled to the free nitrogen of the NPEG-dimerized and resin-bound peptide by consecutive couplings of the Fmoc-protected linker (e.g. Fmoc-Glu-OtBu, Fmoc-GABA, Fmoc-5-Ava-OH) using coupling reagent and base for activation. Alternatively to coupling reagents, activated ester of Fmoc-protected amino acid linkers can be used. Fmoc groups are subsequently removed by deprotection methods.

The fatty acid is coupled to the linker-dimer conjugate using coupling reagent and base for activation. Alternatively, coupled as activated esters. If fatty acid contains carboxylic groups, in addition to the carboxylic group that reacts with the amine of the linker-dimer conjugate, these can be protected as esters, e.g. as methyl ester.

The fatty acid-linked dimeric ligands can be cleaved from the resin with concomitant side-chain deprotection using acids such as TFA or HCl.

Ester protection groups can be removed by stirring the cleaved products in aqueous base (e.g. NaOH, LiOH) and acetonitrile followed by acidification with TFA or HCl.

The final compound of the present invention is obtained by lyophilization and purification by HPLC or similar chromatographic methods.

In a further embodiment, the synthesis of the compounds of the present invention is performed as defined in example 1.

### EXAMPLES

### Example 1

### Synthesis

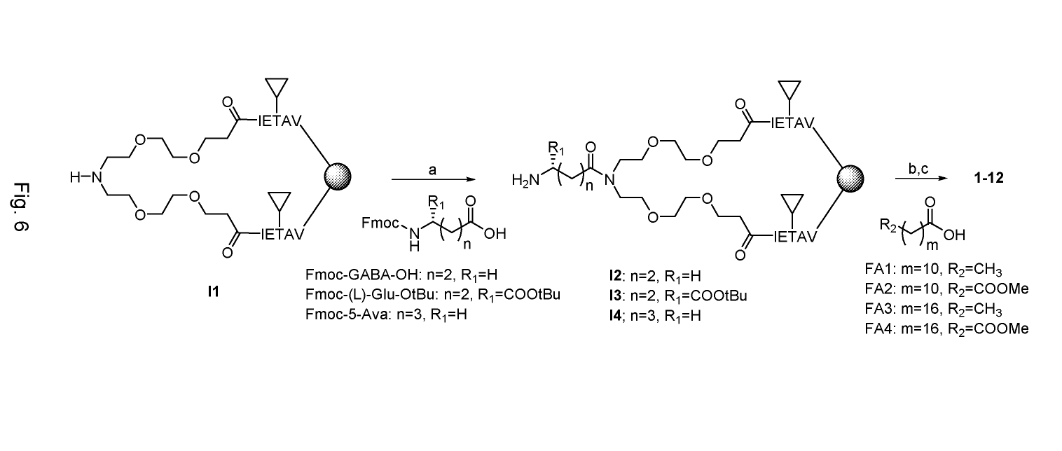

The resin-bound NPEG4 IETAV (SEQ ID NO: 3) dimeric ligand (11) was synthesized as previously described (Bach et al, PNAS USA, 2012, 109, 3317-3322). From this, the appropriately protected linkers (Fmoc-GABA-OH, Fmoc-(L)-Glu-OtBu, and Fmoc-5-Ava, respectively) were attached to the nitrogen in the NPEG4 linker by two subsequent couplings using HATU as the coupling reagent followed by deprotection with piperidine/DMF to give Intermediates I2-4 (FIG. 6). The fatty acids (FA1-4, FIG. 6) were readily attached to the liberated nitrogen in the linkers using solid phase peptide synthesis conditions and cleaved from the resin with concomitant deprotection of the side-chain protecting groups. The terminal methyl protecting group of the mono-protected FA building blocks (dodecanedioic acid methyl ester and octadecanedioic acid methyl ester, FA2 and FA4) were then removed by saponification of the cleaved product followed by acidification (FIG. 6). After lyophilization, the crude products were dissolved in 100% DMSO and purified by large-scale C18 RP-HPLC. The semi-pure fractions (50-90% purity) were lyophilized, re-dissolved in DMSO/ACN/H₂O and purified by preparative C4 RP-HPLC (>95% pure).

FIG. 6 illustrates the synthesis of FA-linked dimeric ligands (1-12). The reaction conditions of scheme 1 was as follows: (a) Fmoc-GABA-OH/Fmoc-(L)-Glu-OtBu/Fmoc-5-Ava, HATU, collidine, DMF (1 h×2), then 20% piperidine in DMF; (b) FA1/FA2/FA3/FA4, HBTU, DIPEA, DMF/DCM, 45 min, then TFA/TIPS/H₂O (90/5/5); (c) 0.5M LiOH, H₂O/ACN (75/25), 30 min, then TFA to pH<2. Triangle indicates that E and T are side-chain protected (tert-butyl).

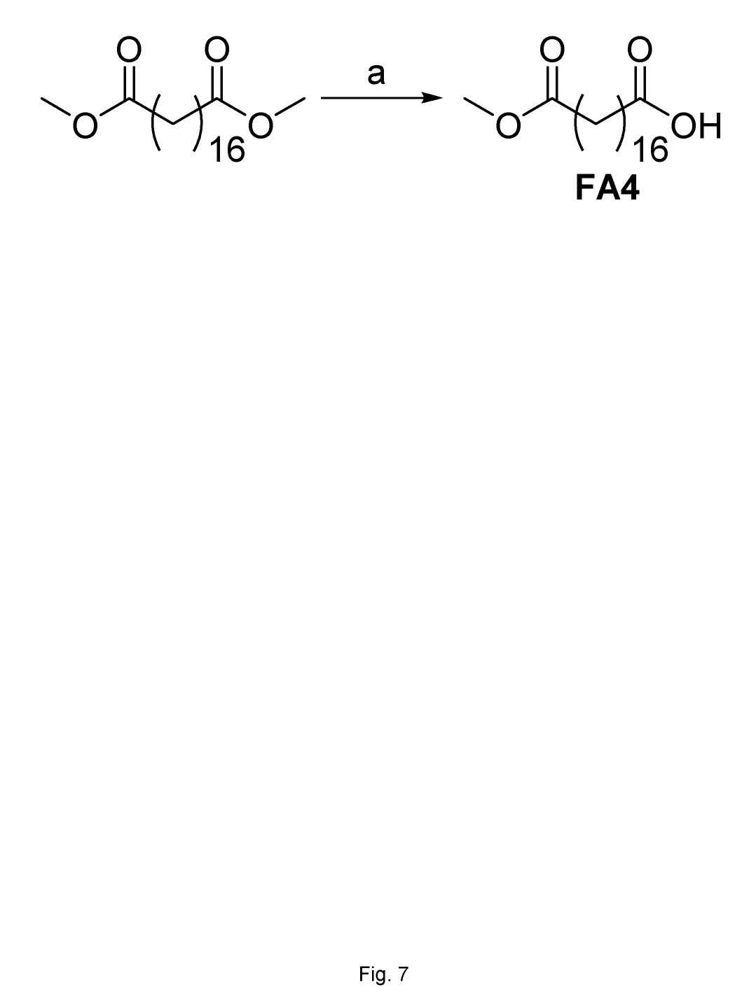

The octadecanedioate monomethyl ester (FA4) was not commercially available and was synthesized by mono-saponification of the corresponding dimethyl ester with one eq. of NaOH as described previously (Jonassen et al, Pharm Res, 2012, 29, 2104-2114) (FIG. 7).

In conclusion, example 1 demonstrates that the compounds of the present invention can be synthesized and obtained in pure form.

### Example 2

### Method for Determining Affinity to HSA

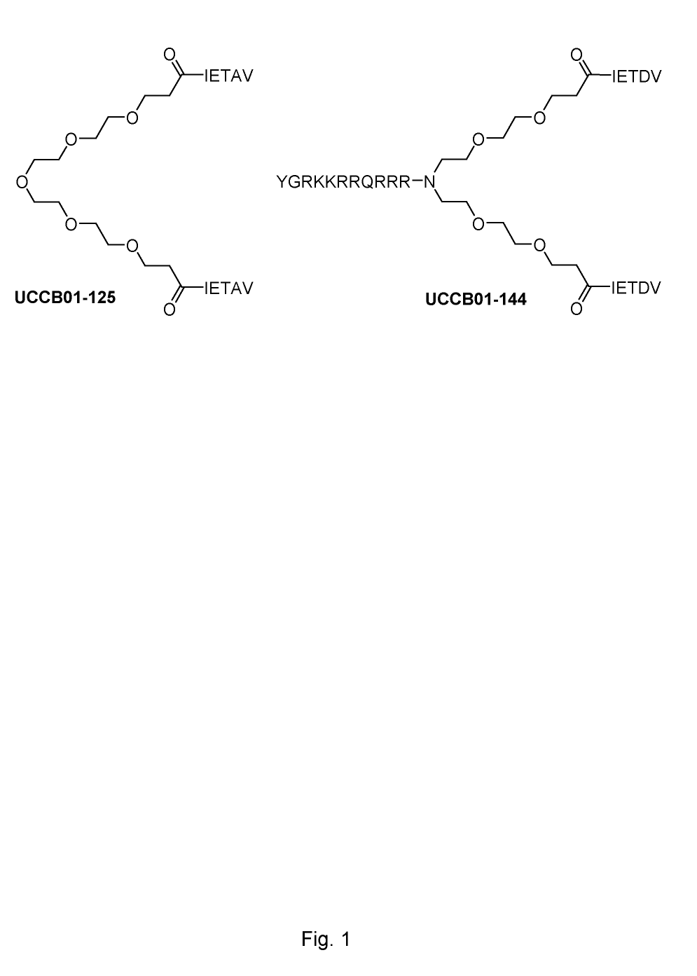

The synthesized FA-linked dimeric ligands (1-12) were evaluated for their HSA affinity using a Transil^{XL} HSA binding assay kit (Sovicell GMBH, Leipzig, Germany). The dimeric ligands, UCCB01-125 (Bach et al, Angew. Chem, Int. Ed, 2009, 48, 9685-9689) and UCCB01-144 (Bach et al, PNAS USA, 2012, 109, 3317-3322) were also tested for comparison (Table 1, FIG. 1).

The assay kit consisted of prefilled wells with increasing concentrations of immobilized HSA as well as two control wells without HSA. The HSA had been immobilized in a random fashion to ensure that all binding sites on HSA were available. To conduct the assay, the wells were incubated with a known concentration of the tested compounds, and the unbound amount of compound was quantified using analytical RP-HPLC (C8 column). The fraction of unbound drug (fᵤ) for each data point was calculated from the RP-HPLC data by comparison to a control sample without HSA. The ratio of HSA-bound drug (f_{b}=1−fᵤ) to unbound drug for each data point was then plotted against the total HSA concentration (c_{HSA}) in the well and fitted to a linear model as given by equation 1, to give the 1/K_{D} as the slope of the fitted curve.

$$\frac{{f}_{b}}{{f}_{u}} = \frac{1}{{K}_{D}} {c}_{HSA} \qquad Equation 1$$

In eq. 1, it is assumed that the concentration of drug-bound HSA ([HSA-D]) is much lower than the total concentration of HSA in the well ([HSA-D]<<c_{HSA}). The assay kit has been designed such that the assumption is valid for compounds where fᵤ>1%. The calculated K_{D}-values were used to calculate f_{b} at physiological concentration of HSA (588 μM) using equation 2.

$${f}_{b} = 1 - \frac{1}{1 + \frac{{c}_{HSA}}{{K}_{D}^{HSA}}} \qquad Equation 2$$

In conclusion, example 3 demonstrates that and how, binding of the compounds of the present invention to human serum albumin (HSA) can be determined.

### Example 3

### Affinity to HSA

Dimeric ligands UCCB01-125 and UCCB01-144, which do not contain FAs, showed HSA affinities of 154.3 μM and 317.5 μM respectively, corresponding to HSA bound fractions (f_{b}) of 75% and 65% respectively (Table 1). However, all FA-linked dimeric ligands (1-12) showed a much higher affinity towards HSA compared to the ligands without FA and accordingly higher f_{b} values (Table 1). Thus, this clearly demonstrates that HSA binding is greatly enhanced as a result of conjugating FA to the dimeric ligands.

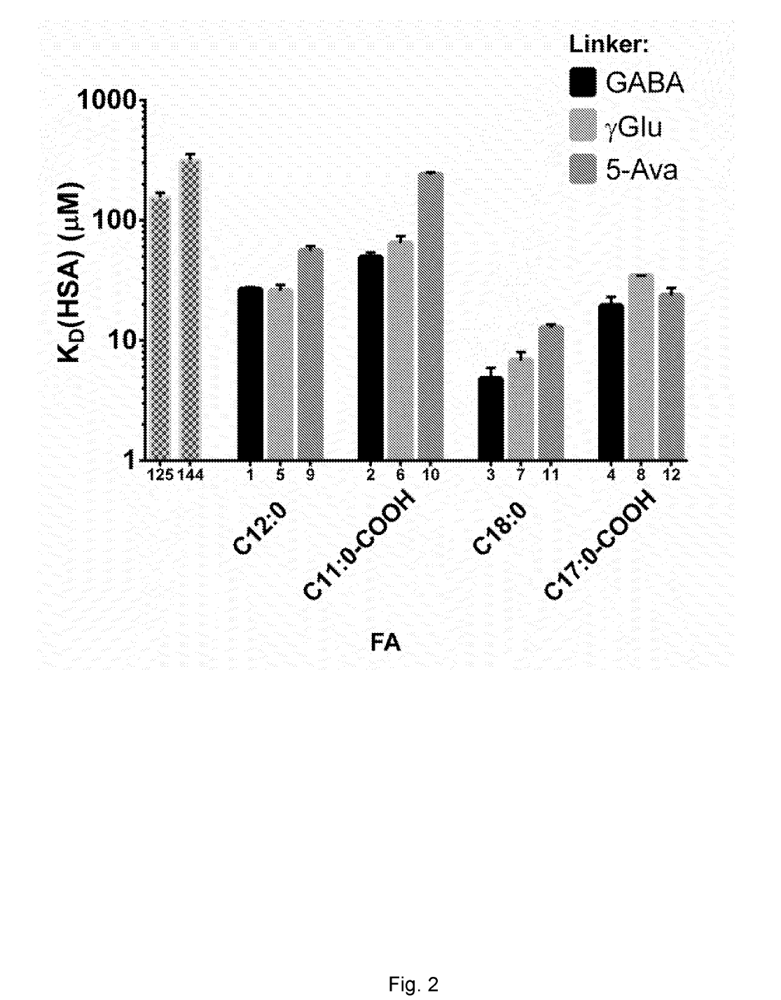

Compounds containing the longer 5-Ava linker generally have slightly lower affinity for HSA than compounds with the shorter linkers (GABA, γGlu) (Table 1, FIG. 2). The additional acid moiety in the γGlu linker (R1) does not seem to have any influence on the affinity for HSA of the FA-linked dimeric ligands synthesized here (Table 1, FIG. 2). This is in contrast to what has been observed in other protein-ligand systems (Hackett et al, Adv Drug Deliv Rev, 2013, 65, 1331-1339) and indicates that the free carboxylate of the γGlu linker is not an essential feature for binding to HSA for the present compounds.

The dimeric ligands linked to the long FAs (m=16) show superior HSA affinity compared to the dimeric ligands linked to the shorter FAs (m=10) (Table 1); and a terminal carboxyl group (R2) has a negative influence on the HSA affinity (Table 1 and FIG. 2).

TABLE 1
HSA binding and calculated fraction of bound compound for FA-linked dimeric ligands (1-12) and
dimeric ligands UCCB01-125 and UCCB01-144^{a}

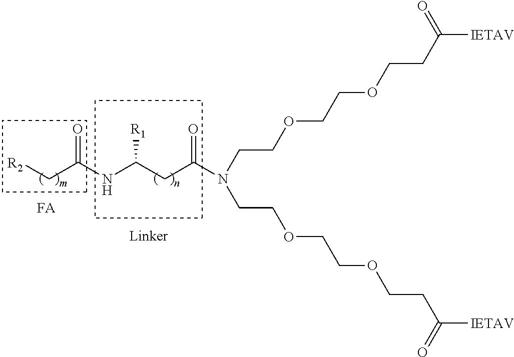

| | | | | | K_{D}(HSA) | |
|---|---|---|---|---|---|---|
| Compound | Linker | FA | m | R₂ | (μM) | f_{b} (%) |
| 1 | GABA | C12:0 | 10 | CH₃ | 26.6 ± 1.1 | 95.7 ± 0.2 |
| | n = 2, R₁ = H | | | | | |
| 2 | | C11:0-COOH | 10 | COOH | 49.3 ± 4.7 | 92.3 ± 0.7 |
| 3 | | C18:0 | 16 | CH₃ | 4.8 ± 1.1 | 99.2 ± 0.2 |
| 4 | | C17:0-COOH | 16 | COOH | 19.4 ± 3.6 | 96.5 ± 0.6 |
| 5 | γGlu, | C12:0 | 10 | CH₃ | 25.8 ± 3.2 | 95.8 ± 0.5 |
| | n = 2, R₁ = COOH | | | | | |
| 6 | | C11:0-COOH | 10 | COOH | 64.6 ± 9.3 | 90.1 ± 1.3 |
| 7 | | C18:0 | 16 | CH₃ | 6.8 ± 1.2 | 98.9 ± 0.2 |
| 8 | | C17:0-COOH | 16 | COOH | 34.3 ± 0.3 | 94.5 ± 0.1 |
| 9 | 5-Ava | C12:0 | 10 | CH₃ | 55.7 ± 5.2 | 91.4 ± 0.7 |
| | n = 3, R₁ = H | | | | | |
| 10 | | C11:0-COOH | 10 | COOH | 240.0 ± 11 | 71.0 ± 1.0 |
| 11 | | C18:0 | 16 | CH₃ | 12.7 ± 0.9 | 97.9 ± 0.2 |
| 12 | | C17:0-COOH | 16 | COOH | 23.5 ± 3.9 | 96.2 ± 0.6 |
| UCCB01-125 | — | — | — | — | 154.3 ± 15.8 | 77.6 ± 1.8 |
| UCCB01-144 | — | — | — | — | 317.5 ± 38.6 | 65.3 ± 2.9 |

^{a}Data shown as mean ± SEM, n = 3

In conclusion, example 3 demonstrates that the compounds of the present invention have an increased affinity for HSA as compared to non-FA derivatized dimeric reference peptides.

### Example 4

### Method for Determining Affinity to PDZ1-2 of PSD-95

Affinity to PSD-95 was measured using an in vitro fluorescence polarization (FP) assay as described by Bach et al (PNAS USA, 2012, 109, 3317-3322). First, a saturation binding curve was obtained to determine K_{D} values for the interaction between a dimeric fluorescent probe and PSD-95 PDZ1-2. Increasing concentrations of PDZ1-2 were added to a constant concentration (0.5 nM) of the probe. The fluorescence polarization of the samples was measured at excitation/emission wavelengths of 635/670 nm and the FP values were fitted to a one site binding model using the program GraphPad Prism. Then, the affinity between the non-fluorescent dimeric ligands and PDZ1-2 were determined in a heterologous competition binding assay, where increasing concentration of ligand was added to a fixed concentration of dimeric probe (0.5 nM) and PDZ1-2 (4 nM). The FP values were fitted to a one site competition (variable slope) model in GraphPad Prism. The resulting IC₅₀ were converted to competition inhibition constants, Kᵢ values, as described (Nikolovska-Coleska et al, Anal Biochem, 2004, 332, 261-273). The modified FP assay was conducted as described above with 1% HSA in the assay.

In conclusion, example 4 demonstrates how to test binding of the compounds of the present invention, to PDZ1-2 of PSD-95.

### Example 5

### Affinity to PDZ1-2 of PSD-95

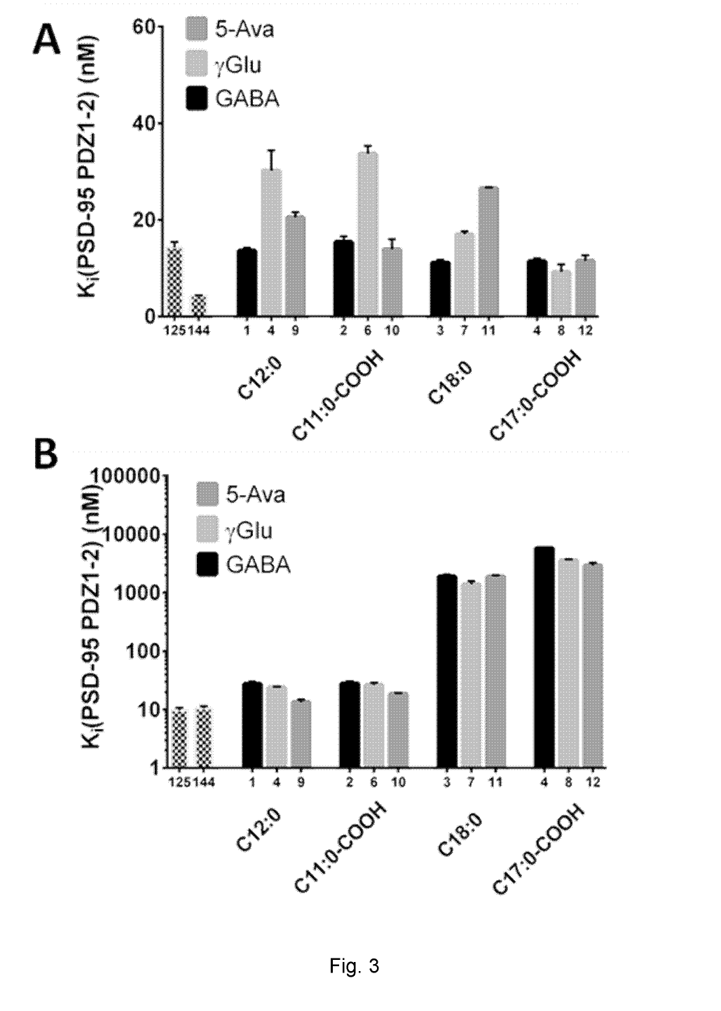

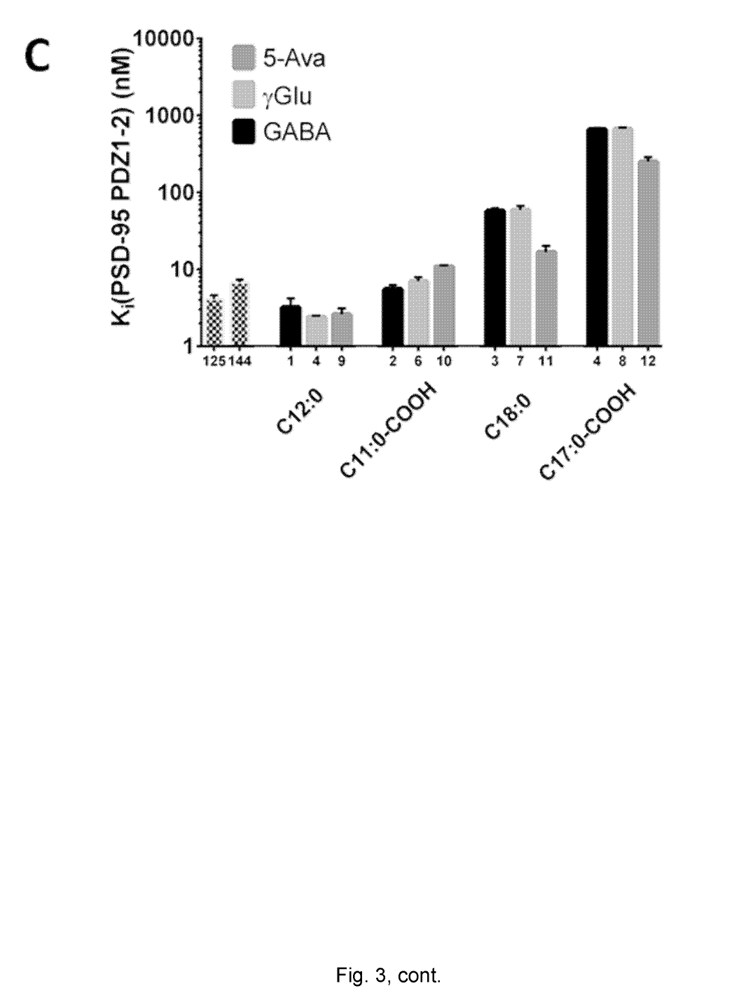

The synthesized FA-linked dimeric ligands were evaluated for their affinity to PSD-95 PDZ1-2 in the FP assay. UCCB01-125 and UCCB01-144 were used as reference compounds (Bach et al, PNAS USA, 2012, 109, 3317-3322) (Table 2, FIG. 3). We first measured the affinities using a simple tris-buffered saline (TBS) buffer (Table 2, FIG. 3A); but furthermore, we investigated if HSA influenced the ability of the FA-linked dimers to bind PSD95 PDZ1-2 by conducting the FP assay with HSA present in the assay buffer (Table 2, FIG. 3B). Due to binding of the probe to HSA at higher concentrations, the concentration of HSA was here set to 1% (˜150 μM), approximately 4 times lower than the estimated physiological blood concentration (520-830 μM) (Kragh-Hansen et al, Biol Pharm Bull 2002, 25, 695-704).

For a traditional small-molecule drug, it is commonly accepted that the unbound fraction of the drug is free to diffuse across membranes and exerts the physiological effect by interacting with its target (Berezhkovskiy et al, J Pharm Sci 2007, 96, 249-257). I.e. if the drug is bound to another molecule, then it cannot interact with the target at the same time. To account for this, the fraction of unbound drug (fu) was calculated from equation 2 (fu=1-fb) at a HSA concentration of 150 μM, and the FP assay data (TBS+HSA) were corrected for the calculated fu (Table 2, FIG. 3C).

TABLE 2
Affinity for PSD-95 PDZ1-2 of FA-linked dimeric ligands (1-12) and dimeric ligands (UCCB01-125 and UCCB01-144)
as determined by FP^{a}, calculated fraction of unbound drug (fᵤ)^{b}, fᵤ-corrected FP data^{a} and retention time (Rₜ) of
the compounds determined by RP-HPLC (C8 column)^{c}.

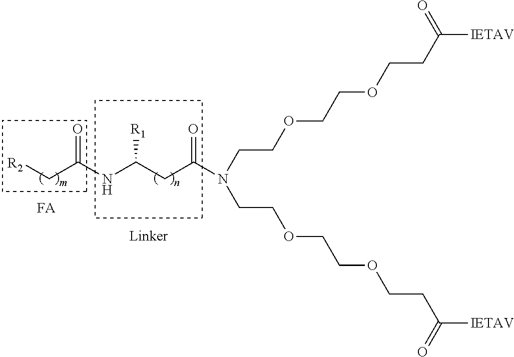

| | | | | | | | | Kᵢ(PSD95), | |
|---|---|---|---|---|---|---|---|---|---|
| | | | | | Kᵢ(PSD95) | Kᵢ(PSD95) + | fᵤ | free ligand | Rₜ |
| Compound | Linker | FA | m | R₂ | (nM) | HSA (nM) | (%) | (nM) | (min) |
| 1 | GABA | C12:0 | 10 | CH₃ | 13.6 ± 0.6 | 27.2 ± 2.9 | 15.1 | 3.2 ± 1.0 | 46 |
| | n = 2, R₁ = H | | | | | | | | |
| 2 | | C11:0-COOH | 10 | COOH | 15.4 ± 1.2 | 27.9 ± 2.7 | 24.7 | 5.5 ± 0.7 | 38 |
| 3 | | C18:0 | 16 | CH₃ | 11.1 ± 0.6 | 1889 ± 155 | 3.1 | 57.2 ± 4.9 | 61 |
| 4 | | C17:0-COOH | 16 | COOH | 11.4 ± 0.6 | 5717 ± 298 | 11.5 | 654 ± 34 | 48 |
| 5 | γGlu, | C12:0 | 10 | CH₃ | 30.2 ± 4.2 | 24.0 ± 0.6 | 14.7 | 2.4 ± 0.1 | 46 |
| | n = 2, R₁ = COOH | | | | | | | | |
| 6 | | C11:0-COOH | 10 | COOH | 33.7 ± 1.6 | 26.4 ± 2.6 | 30.1 | 7.0 ± 0.9 | 37 |
| 7 | | C18:0 | 16 | CH₃ | 17.0 ± 0.6 | 1391 ± 184 | 4.3 | 59.1 ± 7.8 | 59 |
| 8 | | C17:0-COOH | 16 | COOH | 9.2 ± 1.6 | 3594 ± 132 | 18.6 | 669 ± 25 | 47 |
| 9 | 5-Ava | C12:0 | 10 | CH₃ | 20.5 ± 1.1 | 13.4 ± 1.4 | 27.1 | 2.6 ± 0.5 | 48 |
| | n = 2, R₁ = H | | | | | | | | |
| 10 | | C11:0-COOH | 10 | COOH | 13.9 ± 2.1 | 18.5 ± 0.6 | 61.5 | 10.9 ± 0.4 | 38 |
| 11 | | C18:0 | 16 | CH₃ | 26.5 ± 0.2 | 1889 ± 100 | 7.8 | 16.8 ± 3.3 | 61 |
| 12 | | C17:0-COOH | 16 | COOH | 11.5 ± 1.2 | 2926 ± 335 | 8.3 | 250 ± 37 | 49 |
| UCCB01-125 | — | — | — | — | 14.3 ± 1.1 | 9.7 ± 1.0 | 50.7 | 4.1 ± 0.5 | 28 |
| UCCB01-144 | — | — | — | — | 4.3 ± 0.1 | 10.5 ± 0.9 | 67.9 | 6.7 ± 0.6 | 24 |

^{a}FP data recorded in TBS and in TBS with 1% HSA. Data shown as mean ± SEM, n ≧ 3.
^{b}Fᵤ calculated according to equation 2, f_{b} = 1-fᵤ, ^{c}n = 1.

The affinity for PSD-95 PDZ1-2 in TBS was comparable for all of the FA-linked dimeric ligands to the affinities of UCCB01-125 (Table 2, FIG. 3A), showing that the affinity for PSD-95 PDZ1-2 was not influenced by the FA-derivatization.

The affinity for PSD-95 PDZ1-2 in TBS with 1% HSA varied significantly and systematically between the compounds (Table 2, FIG. 3B). The dimeric ligands that were linked to the longer FAs (C18:0 or C17:0-COOH) generally had an apparent >50-fold lower affinity for PSD-95 PDZ1-2 than the dimeric ligands linked to the shorter FAs (C12:0 or C11:0-COOH).

When the FP data were corrected for fu (Table 2, FIG. 3C), a systematic ranking of affinities for PSD-95 PDZ1-2 within each linker series was revealed. The C12:0-linked dimeric ligands (1, 5, 9) had the highest affinity, followed by C11:0-COOH (2, 6, 10), C18:0 (3, 7, 11) and C17:0-COOH (4, 8, 12).

The Fu-corrected FP data also revealed that the observed 50-fold affinity loss of the C18:0-linked dimeric ligands 3, 7 and 11 when the FP measurement was conducted in TBS+HSA was mainly caused by a high binding of the compounds to HSA, although a 4-5 fold decrease in affinity for PSD-95 was seen for 3 and 7, compared to the FP data recorded in TBS (3: Kᵢ=57.2 nM vs 11.1 nM; 7: Kᵢ=59.1 nM vs 17.0 nM, Table 2) in the current case the reduction in affinity was HSA-dependent, since no decrease in affinity was seen in TBS.

The two 5-Ava-linked dimeric ligands 11 and 12 were less affected by HSA than the corresponding GABA and γGlu-linked dimeric ligands (3, 4, 7 and 8).

In conclusion, example 5 demonstrates that the compounds of the present invention bind to PDZ1-2 of PSD-95.

### Example 6

### Hydrophobicity of FA-Linked Dimeric Ligands

The compounds with the highest affinity for HSA (3, 7, 11) were also the most hydrophobic of the synthesized compounds as judged by the retention time (Rₜ) determined by analytical RP-HPLC (Table 2). In an attempt to increase the hydrophilicity and thus solubility of these compounds, analogues of 3 and 7 were made, where the peptide sequence was replaced with IETDV (SEQ ID NO: 4) instead of IETAV (SEQ ID NO: 3) (13, 15; Table 3), as the additional charge introduced by the Asp (D) moiety could increase the hydrophilicity of the dimers. An IETDV analogue of the highest HSA affinity compound containing a terminal acid moiety (4) was also synthesized and tested (14) for comparison. These FA linked dimers were synthesized analogously to 1-12 (FIG. 6) using the appropriate peptide sequence (IETDV) as starting point.

HPLC analysis revealed a minor or no decrease in Rₜ values, and thus hydrophobicity, for IETDV-based compounds (13-15) relative to IETAV-based compounds (3, 4, 7); but a systematic reduction in HSA affinities were seen (Table 3). For example, 13 eluted 3 minutes earlier on the analytical RP-HPLC than the IETAV analogue (13: 58 min, 3: 61 min, Table 3), but the HSA affinity was reduced (13: K_{D}=83.0 μM, 3: K_{D}=4.8 μM, table 3).

TABLE 3
Comparison of FA-linked dimeric analogues with different peptide sequences.
3, 4 and 7 peptide sequence IETAV (SEQ ID NO: 3); 13, 14 and 15 peptide
sequence IETDV (SEQ ID NO: 4).

| | | | | | | | Kᵢ(PSD- | |
|---|---|---|---|---|---|---|---|---|
| | | | | | Kᵢ(PSD- | | 95), free | |
| | | | K_{D}(HSA) | Kᵢ(PSD- | 95) + HSA | fᵤ | ligand | Rₜ |
| Compound | Linker | FA | (μM) | 95) (nM) | (nM) | (%) | (nM) | (min) |
| 3 | GABA | C18:0 | 4.8 ± 1.1 | 11.1 ± 0.6 | 1889 ± 155 | 3.1 | 57.2 ± 4.9 | 61 |
| 4 | GABA | C17:0-COOH | 19.4 ± 3.6 | 11.4 ± 0.6 | 5717 ± 298 | 11.5 | 654 ± 34 | 48 |
| 7 | γGlu | C18:0 | 6.8 ± 1.2 | 17.0 ± 0.6 | 1391 ± 184 | 4.3 | 59.1 ± 7.8 | 59 |
| 13 | GABA | C18:0 | 83.0 ± 11.0 | 8.0 ± 0.8 | 2514 ± 149 | 35.6 | 896 ± 53 | 58 |
| 14 | GABA | C17:0-COOH | 116.0 ± 7.6 | 29.3 ± 0.7 | 770 ± 27 | 43.6 | 1097 ± 65 | 47 |
| 15 | γGlu | C18:0 | 55.0 ± 3.4 | 6.7 ± 0.6 | 3806 ± 287 | 26.8 | 1022 ± 77 | 59 |

In conclusion, example 6 demonstrates that affinity for HSA is dependent not only on the fatty acid of choice, but also on the peptide sequence elected.

### Example 7

### Plasma Stability of Compound 1, 4, 7 and 13)

The plasma-stability of 1, 4, 7 and 13 was evaluated in a modified version of an in vitro plasma stability assay (Bach et al, Angew. Chem, Int. Ed, 2009, 48, 9685-9689). In the original procedure, the investigated compound was incubated in human plasma. Samples were then taken out at appropriate timepoints and the serum proteins were removed by precipitation with trichloroacetic acid (TCA) followed by analysis of the supernatants by RP-HPLC. The obtained peak areas were normalized to the amount at T₀ and fitted to a 1st order decay model to calculate the half-life. When this method was applied to the FA-linked dimeric ligands, sample recoveries were low (<5%). This was caused by the removal of the FA-linked dimeric ligands as HSA-bound complexes during the TCA precipitation. Therefore dissolution of the sample in solid guanidine hydrochloride (GnHCI) to a final concentration of 6M was performed prior to the TCA precipitation. The purpose of this was to unfold the HSA in the sample, releasing the FA-linked dimeric ligand.

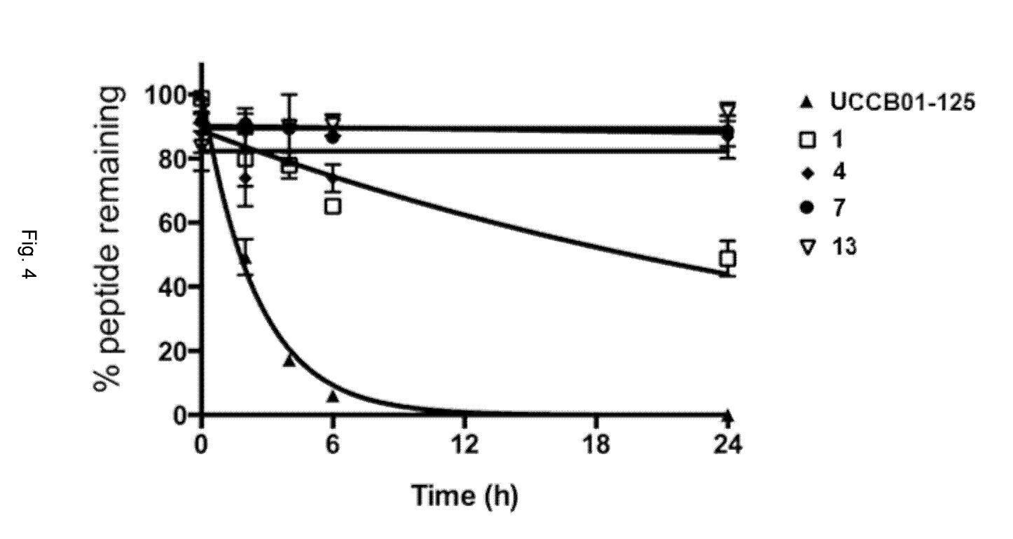

All of the FA-linked dimeric ligands were more stable in the in-vitro plasma stability assay than UCCB01-125 (FIG. 4). Without being bound by theory, it is expected that this is due to the higher HSA binding of the compounds, lowering the free concentration of compound available for enzymatic digestion. The compound containing the shorter C12:0 FA (1) was degraded faster than the compounds containing the longer C18:0 or C17:0-COOH FA (4, 7, 13), which were highly stable. The prolonged stability is explained by the increased affinity to HSA, which prevent proteases from cleaving the dimeric peptide-based compounds, and steric hindrance mediated by the FA.

In conclusion, example 7 demonstrates a method of assessing blood plasma stability in vitro, and that the FA linked dimeric compounds of the present invention have increased plasma stability and half-life as compared to non-FA linked reference compounds.

### Example 8

### In Vivo Pharmacokinetic Studies

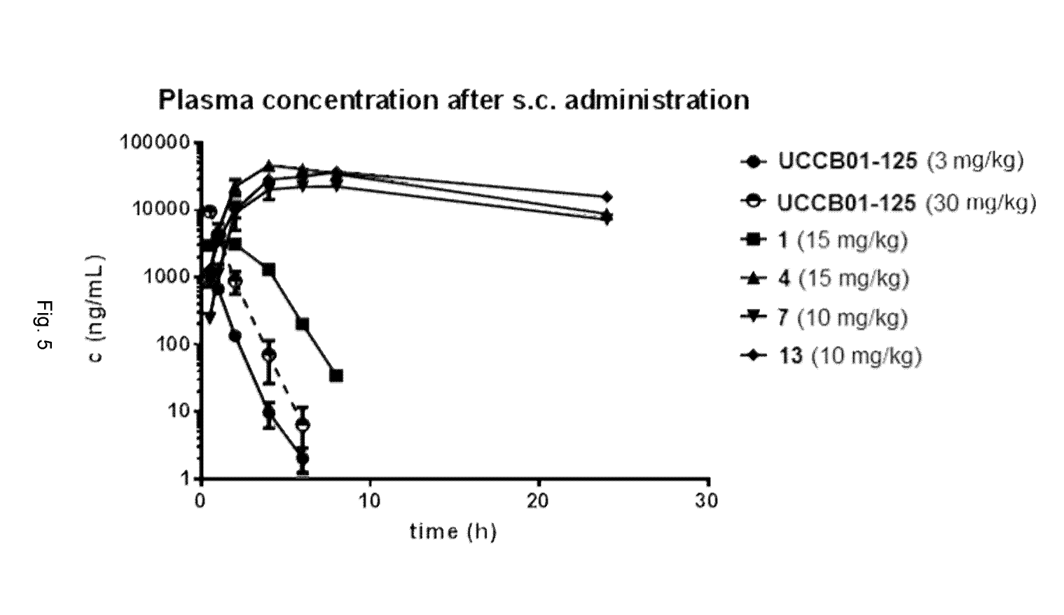

To determine the pharmacokinetic properties of FA-linked compounds we measured the concentration of selected compounds in blood by LC-MS/MS following a single subcutaneous (s.c.) bolus injection in male Wistar rats (FIG. 5). From this, it was apparent that all FA-linked dimeric ligands have longer T_{1/2} and greater Tₘₐₓ than dimeric ligand without FA (UCCB01-125) (Table 4 and FIG. 5). The effect was smallest for 1, but very noticeable for 4, 7, and 13 which showed T_{1/2} greater than 8 hours, corresponding to a >16-fold increase relative to UCCB01-125. The increased Tₘₐₓ is explained by a prolonged absorption from the injection site. Overall, these properties enable administration by s.c.depot injections and thereby slow and consistent release of compound into the blood, whereby fewer administrations are needed to maintain pharmaceutical relevant blood concentrations.

TABLE 4
Pharmacokinetic parameters of FA-linked dimeric ligands after s.c. injection in rats

| | | | Peptide | Dose | | |
|---|---|---|---|---|---|---|
| Compound | Linker | FA | sequence | (mg/kg) | T_{1/2} (h)^{a} | Tₘₐₓ (h)^{a} |
| UCCB01-125 | — | — | IETAV | 3 | 0.561 ± 0.101 | 0.5 ± 0 |
| | | | | 30 | 0.450 ± 0.068 | 0.5 ± 0 |
| 1 | GABA | C12 | IETAV | 15 | 0.768 ± 0.045 | 0.833 ± 0.167 |
| 4 | GABA | C17:0-COOH | IETAV | 15 | 8.13 ± 0.50 | 4.67 ± 0.66 |
| 7 | γGlu | C18:0 | IETAV | 10 | 10.7 ± 0.58 | 6.00 ± 1.15 |
| 13 | GABA | C18:0 | IETDV | 10 | 16.3 ± 2.81 | 8.00 ± 0 |

^{a}Data given as mean ± SEM, n = 3.

### Example 9

### Sequences

SEQ ID NO: 1
X₄X₃X₂X₁

wherein
X₄ is an amino acid residue selected from E, Q, A, N and S,
X₃ is an amino acid residue selected from S and T,
X₂ is an amino acid residue selected from A, D, E, Q, N, S, V, N-Me-A, N-Me-D, N-Me-E, N-Me-Q, N-Me-N, N-Me-S and N-Me-V
X₁ is an amino acid residue selected from I, L and V

SEQ ID NO: 2
Z₄Z₃Z₂Z₁

wherein
Z₄ is an amino acid residue selected from E, Q, A, N and S,
Z₃ is an amino acid residue selected from S and T,
Z₂ is an amino acid residue selected from A, D, E, Q, N, S, V, N-Me-A, N-Me-D, N-Me-E, N-Me-Q, N-Me-N, N-Me-S and N-Me-V
Z₁ is an amino acid residue selected from I, L and V

SEQ ID NO: 3
IETAV

SEQ ID NO: 4
IETDV
SEQ ID NO: 5
X₅X₄X₃X₂X₁

wherein
X₅ is any amino acid residue,
X₄ is an amino acid residue selected from E, Q, A, N and S,
X₃ is an amino acid residue selected from S and T,
X₂ is an amino acid residue selected from A, D, E, Q, N, S, V, N-Me-A, N-Me-D, N-Me-E, N-Me-Q, N-Me-N, N-Me-S and N-Me-V
X₁ is an amino acid residue selected from I, L and V

SEQ ID NO: 6
Z₅Z₄Z₃Z₂Z₁

wherein
Z₅ is any amino acid residue,
Z₄ is an amino acid residue selected from E, Q, A, N and S,
Z₃ is an amino acid residue selected from S and T,
Z₂ is an amino acid residue selected from A, D, E, Q, N, S, V, N-Me-A, N-Me-D, N-Me-E, N-Me-Q, N-Me-N, N-Me-S and N-Me-V
Z₁ is an amino acid residue selected from I, L and V

1. A compound comprising a first peptide (P₁) and a second peptide (P₂), wherein P₁ and P₂ individually comprise at least two proteinogenic or non-proteinogenic amino acid residues, and wherein both P₁ and P₂ are conjugated to a first linker L₁ via their N-termini, and wherein L₁ comprises polyethylene glycol (PEG) wherein at least one oxygen atom of said PEG is substituted with a nitrogen atom to give NPEG, and wherein an albumin binding moiety is linked to the nitrogen atom of the NPEG by an amide bond, or via an optional linker L₂.

2. The compound according to claim 1, wherein said compound has the generic structure of formula (I):

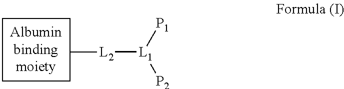

3. The compound according to any one of the preceding claims, wherein the albumin binding moiety is a fatty acid (FA).

4. The compound according to any one of the preceding claims, wherein said compound has the generic structure of formula (II):

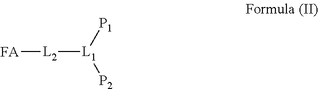

5. The compound according to any one of the preceding claims, wherein the fatty acid is a saturated or unsaturated fatty acid.

6. The compound according to any one of the preceding claims, wherein the fatty acid is linked to the nitrogen atom of the NPEG linker (L₁) via a second linker L₂, wherein L₂ comprises a nitrogen atom.

7. The compound according to any one of the preceding claims, wherein the second linker L₂ comprises one or more moieties selected from the group consisting of γ-Glu, γ-butyric acid (GABA), 5-amino valeric acid (5-Ava), proteinogenic amino acids, non-proteinogenic amino acids, and any compound having the general formula H₂N-[Q]-COOH, wherein Q is any suitable atom or atoms.

8. The compound according to any one of the preceding claims, wherein said compound has the generic structure of formula (III) or (IV): 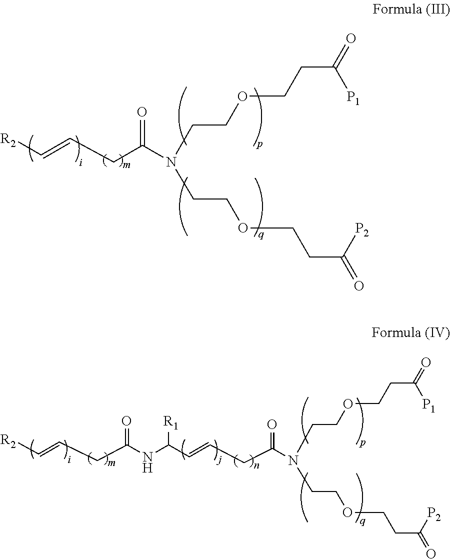
wherein
R1 individually are selected from the group consisting of H and COOH,
n is an integer 0 to 48,
m is an integer 1 to 48,
p is an integer 0 to 28,
q is an integer 0 to 28,
i is an integer 0 to 12,
j is an integer 0 to 12
P₁ and P₂ are individually selected from peptides comprising at least two proteinogenic or non-proteinogenic amino acid residues.

9. The compound according to any one of the preceding claims, wherein p=q.

10. The compound according to any one of the preceding claims, wherein p>q.

11. The compound according to any one of the preceding claims, wherein p<q.

12. The compound according to any one of the preceding claims, wherein the sum of p and q is an integer between 1 and 28.

13. The compound according to any one of the preceding claims, wherein the number of ethylene glycol moieties, p is selected from 0, 1, 2, 3, 4, 5, 6, 7, 8, 9, 10, 11, 12, 13, 14, 15, 16, 17, 18, 19, 20, 21, 22, 23, 24, 25, 26, 27 or 28.

14. The compound according to any one of the preceding claims, wherein the number of ethylene glycol moieties, p, is 0 to 4.

15. The compound according to any one of the preceding claims, wherein the number of ethylene glycol moieties, q is selected from 0, 1, 2, 3, 4, 5, 6, 7, 8, 9, 10, 11, 12, 13, 14, 15, 16, 17, 18, 19, 20, 21, 22, 23, 24, 25, 26, 27 or 28 ethylene glycol moieties.

16. The compound according to any one of the preceding claims, wherein the number of ethylene glycol moieties, q, is 0 to 4.

17. The compound according to any one of the preceding claims, wherein the total number of ethylene glycol moieties p+q is between 2 and 12.

18. The compound according to any one of the preceding claims, wherein the total number of ethylene glycol moieties p+q is 4.

19. The compound according to any one of the preceding claims, wherein the total number of ethylene glycol moieties p+q is 6.

20. The compound according to any one of the preceding claims, wherein n is an integer selected from the group consisting of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 and 48.

21. The compound according to any one of the preceding claims, wherein n is an integer between 1 and 3.

22. The compound according to any one of the preceding claims, wherein n=1.

23. The compound according to any one of the preceding claims, wherein n=2.

24. The compound according to any one of the preceding claims, wherein n=3.

25. The compound according to any one of the preceding claims, wherein m is an integer selected from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 and 48.

26. The compound according to any one of the preceding claims, wherein m is an integer between 10 and 16.

27. The compound according to any one of the preceding claims, wherein m=10.

28. The compound according to any one of the preceding claims, wherein m=16.

29. The compound according to any one of the preceding claims, wherein the fatty acid is a C₄-C₂₂ fatty acid.

30. The compound according to any one of the preceding claims, wherein the fatty acid is selected from the group consisting of caprylic acid, capric acid, lauric acid, myristic acid, palmitic acid, stearic acid, arachidic acid, behenic acid, lignoceric acid and cerotic acid.

31. The compound according to any one of the preceding claims, wherein the fatty acid is selected from the group consisting of myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid and docosahexaenoic acid.

32. The compound according to any one of the preceding claims, wherein
P₁ comprises the amino acid sequence X₄X₃X₂X₁ (SEQ ID NO: 1), and
P₂ comprises the amino acid sequence Z₄Z₃Z₂Z₁ (SEQ ID NO: 2),
wherein
a) X₁ and/or Z₁ is an amino acid residue selected from I, L and V,
b) X₂ and/or Z₂ is an amino acid residue selected from A, D, E, Q, N, S, V, N-Me-A, N-Me-D, N-Me-E, N-Me-Q, N-Me-N, N-Me-S and N-Me-V,
c) X₃ and/or Z₃ is an amino acid residue selected from S and T,
d) X₄ and/or Z₄ is an amino acid residue selected from E, Q, A, N and S,
wherein X₁ and Z₁ both individually represent the ultimate C-terminal amino acid residue comprising a free carboxylic acid.

33. The compound according to any one of the preceding claims, wherein said compound has the generic structure of formula (V) or (VI): 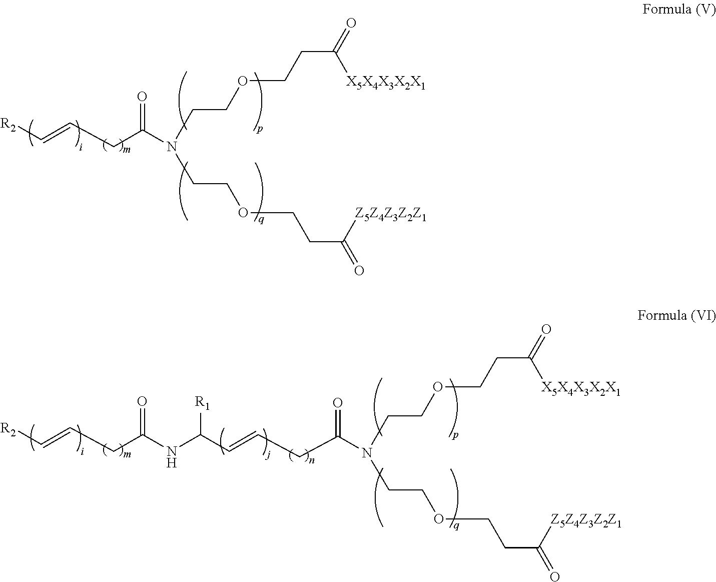
wherein
R₁ and R₂ individually are selected from the group consisting of H and COOH,
n is an integer 0 to 48,
m is an integer 1 to 48, and
p is an integer 0 to 28,
q is an integer 0 to 28,
i is an integer 0 to 12,
j is an integer 0 to 12
X₅ and/or Z₅ are/is an optional amino acid residue, a peptide or a polypeptide,
X₄ and/or Z₄ is an amino acid residue selected from E, Q, A, N and S,
X₃ and/or Z₃ is an amino acid residue selected from S and T,
X₂ and/or Z₂ is an amino acid residue selected from A, D, E, Q, N, S, V, N-Me-A, N-Me-D, N-Me-E, N-Me-Q, N-Me-N, N-Me-S and N-Me-V
X₁ and/or Z₁ is an amino acid residue selected from I, L and V.

34. The compound according to any one of the preceding claims, wherein X₅ is a proteinogenic or a non-proteinogenic amino acid residue.

35. The compound according to any one of the preceding claims, wherein X₅ is an amino acid residue selected from the group consisting of I, A, L and V.

36. The compound according to any one of the preceding claims, wherein X₅ is a peptide or polypeptide having an amino acid sequence consisting of between 2 to 100 amino acid residues, wherein the C terminus of said peptide or polypeptide is an amino acid residue selected from the group consisting of I, A, L and V.

37. The compound according to any one of claims 2 and 3, wherein X₅ is a peptide comprising 2 to 100 residues, such as 2 to 90 amino acid residues, such as 2 to 80 amino acid residues, such as 2 to 70 amino acid residues, such as 2 to 60 amino acid residues, such as 2 to 50 amino acid residues, such as 2 to 40 amino acid residues, such as 2 to 30 amino acid residues, such as 2 to 20 amino acid residues, such as 2 to 10 amino acid residues, such as 2 to 9 amino acid residues, such as 2 to 8 amino acid residues, such as 2 to 7 amino acid residues, such as 2 to 6 amino acid residues, such as 2 to 5 amino acid residues, such as 2 to 4 amino acid residues, such as 2 to 3 amino acid residues,
wherein the C terminus is an amino acid selected from the group consisting of I, A, L and V

38. The compound according to any one of the preceding claims, wherein the compound is selected from the group consisting of:

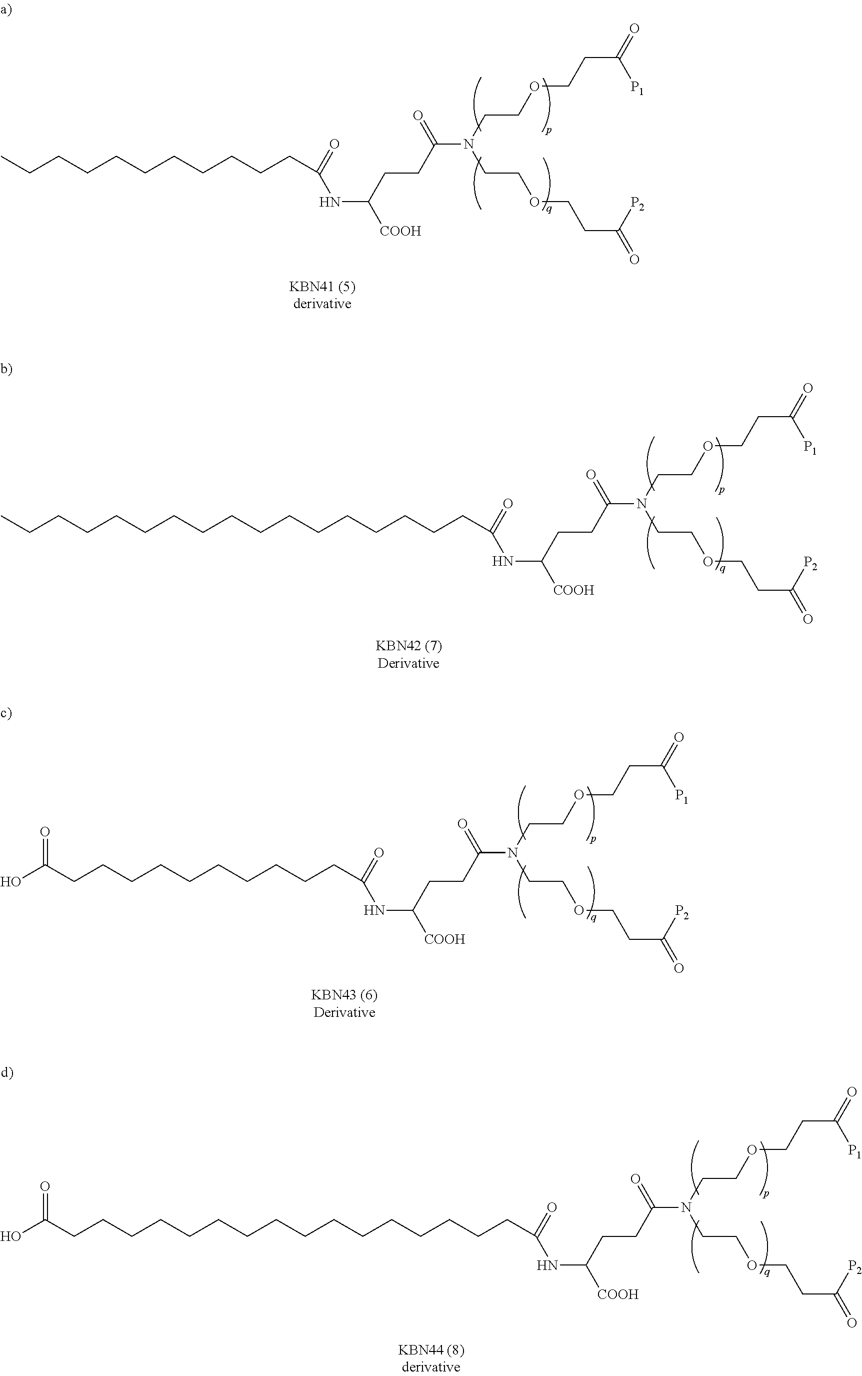

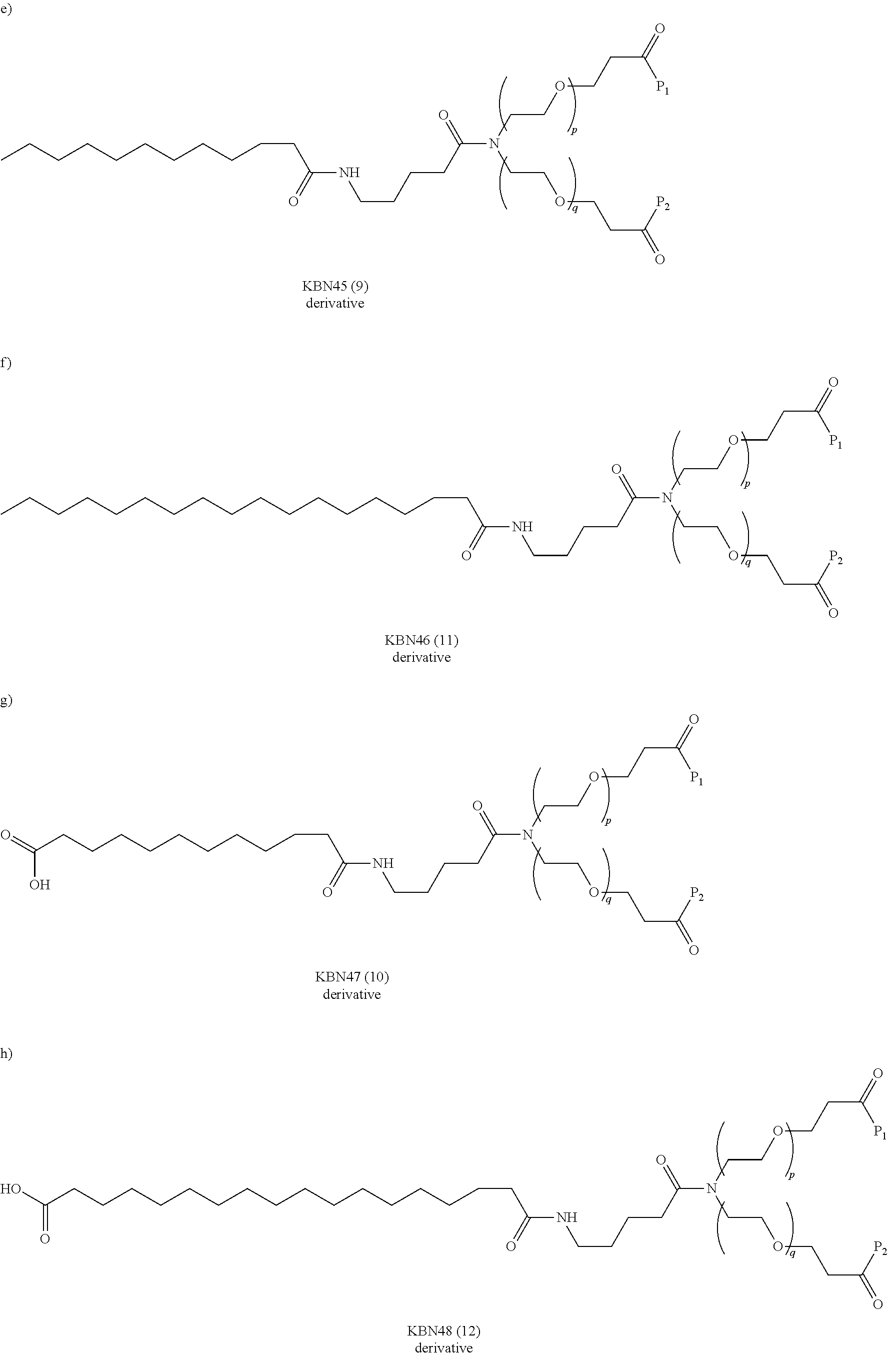

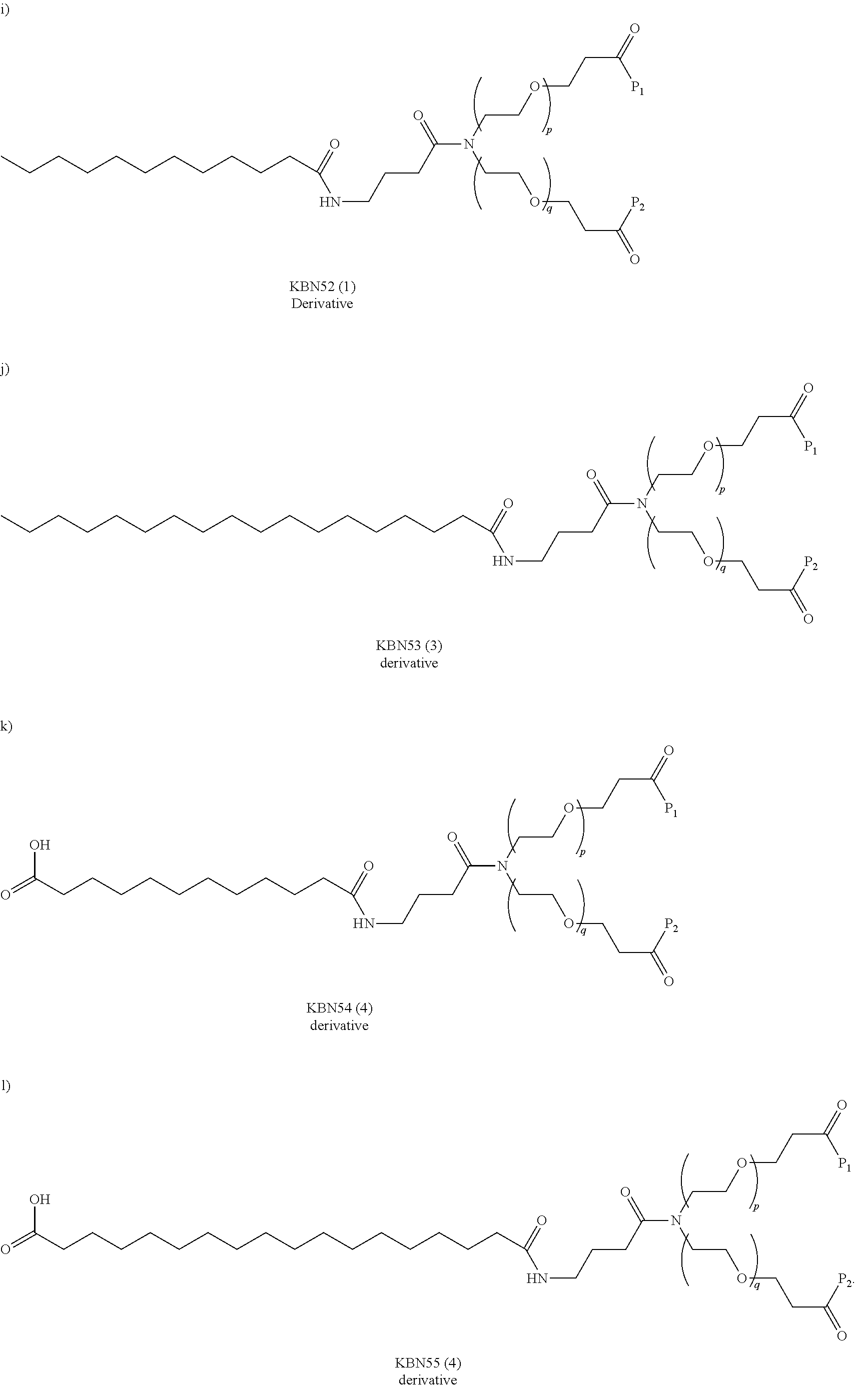

39. The compound according to any one of the preceding claims, wherein the compound is selected from the group consisting of:

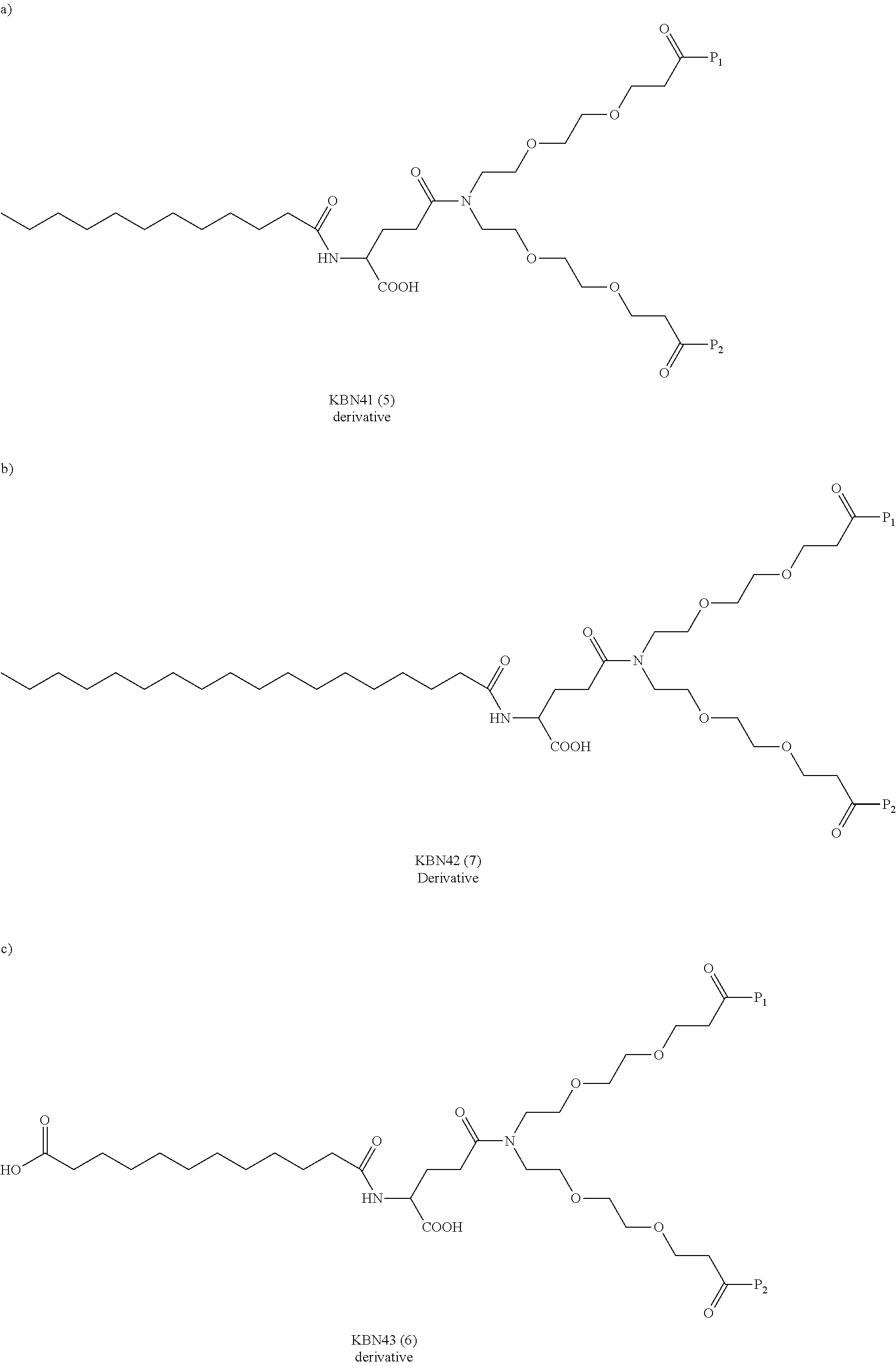

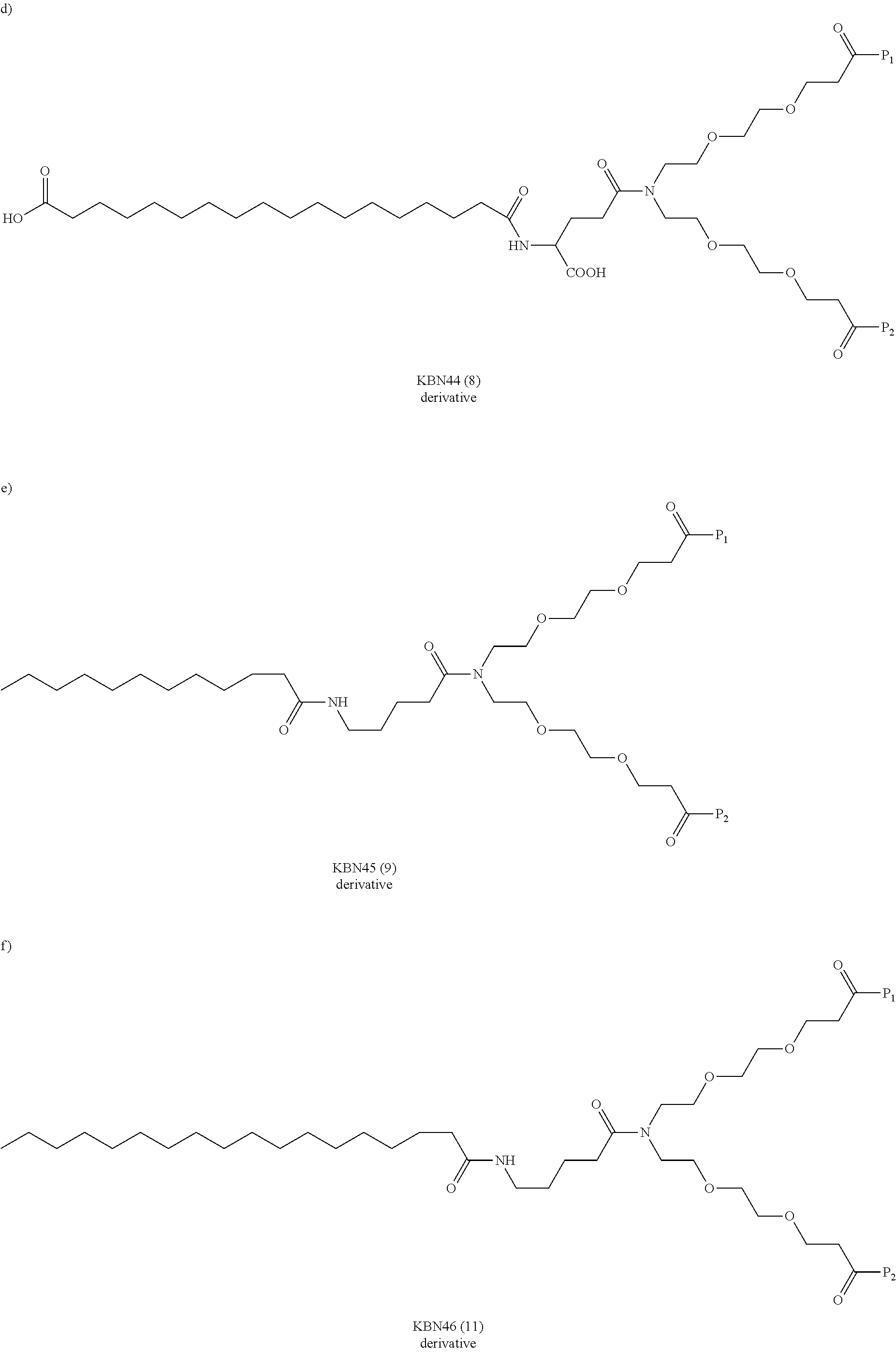

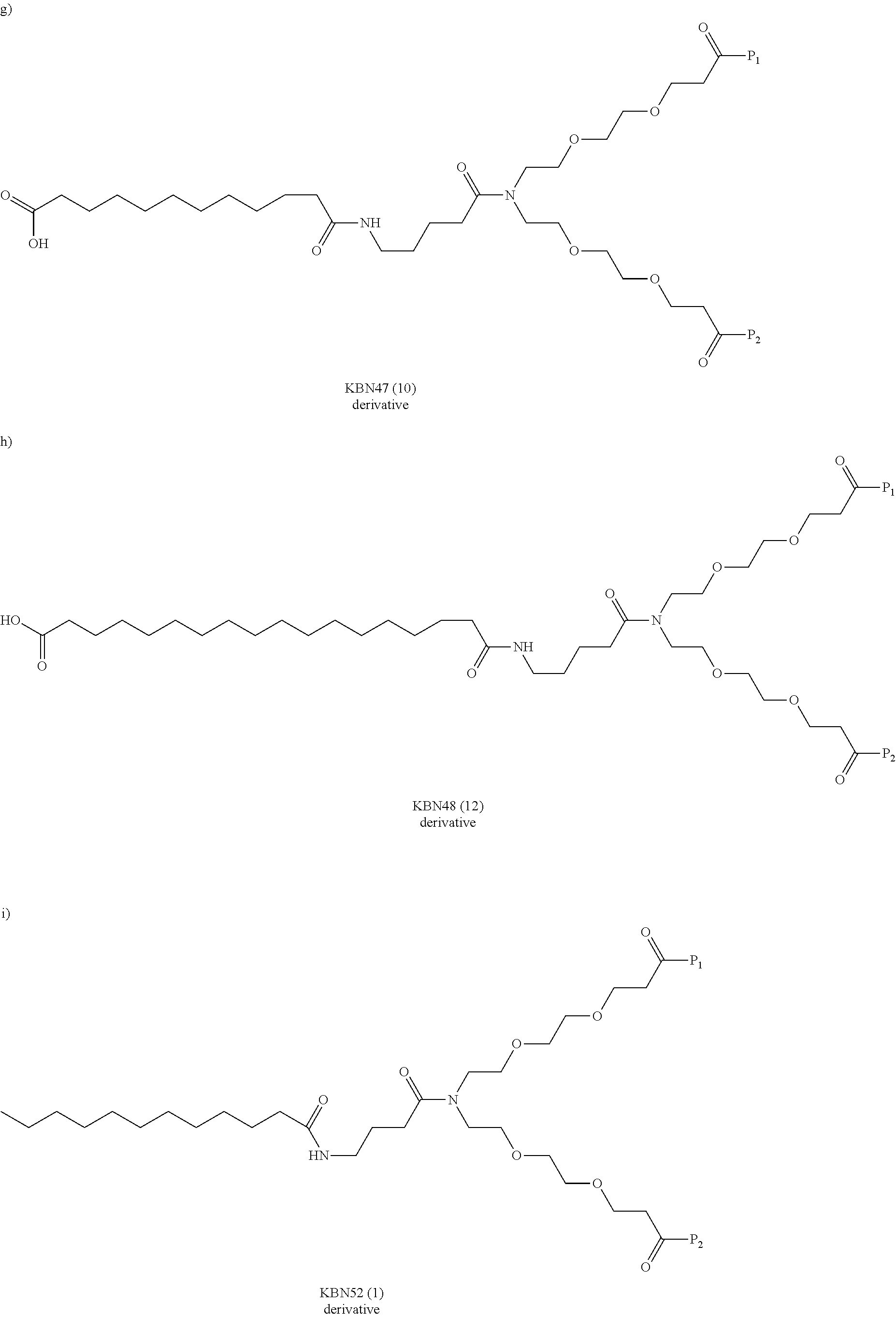

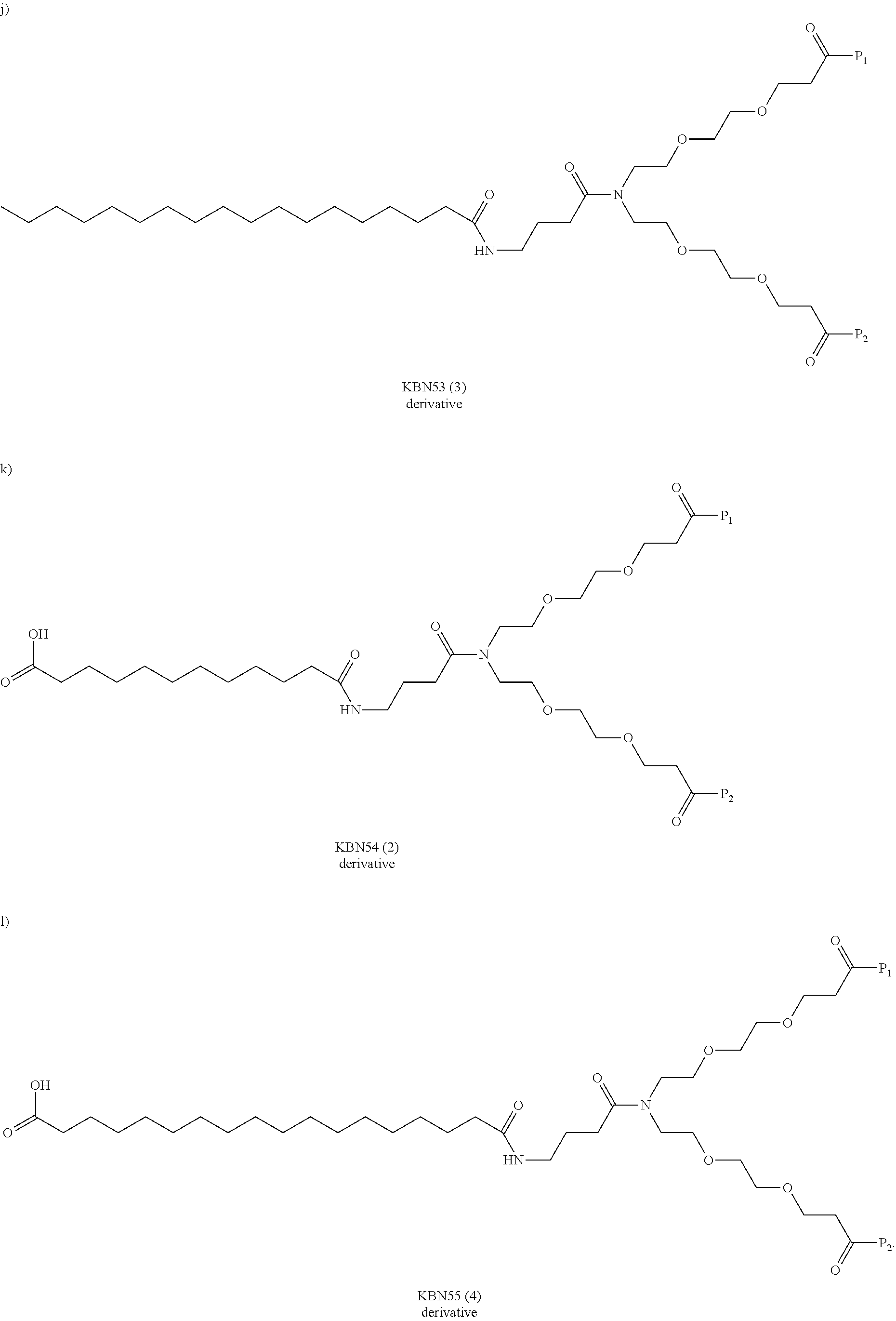

40. The compound according to any one of the preceding claims, wherein the compound is selected from the group consisting of:

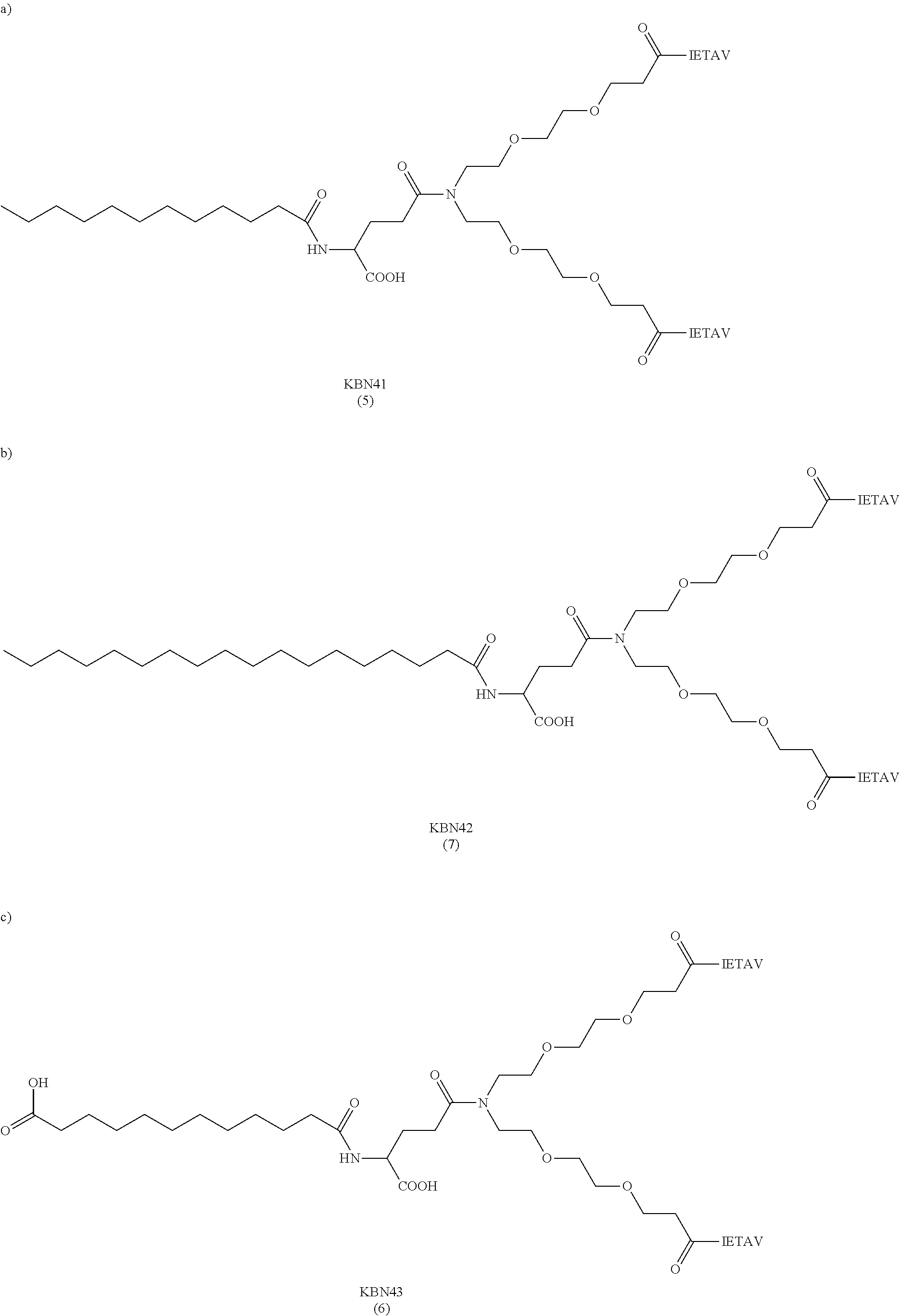

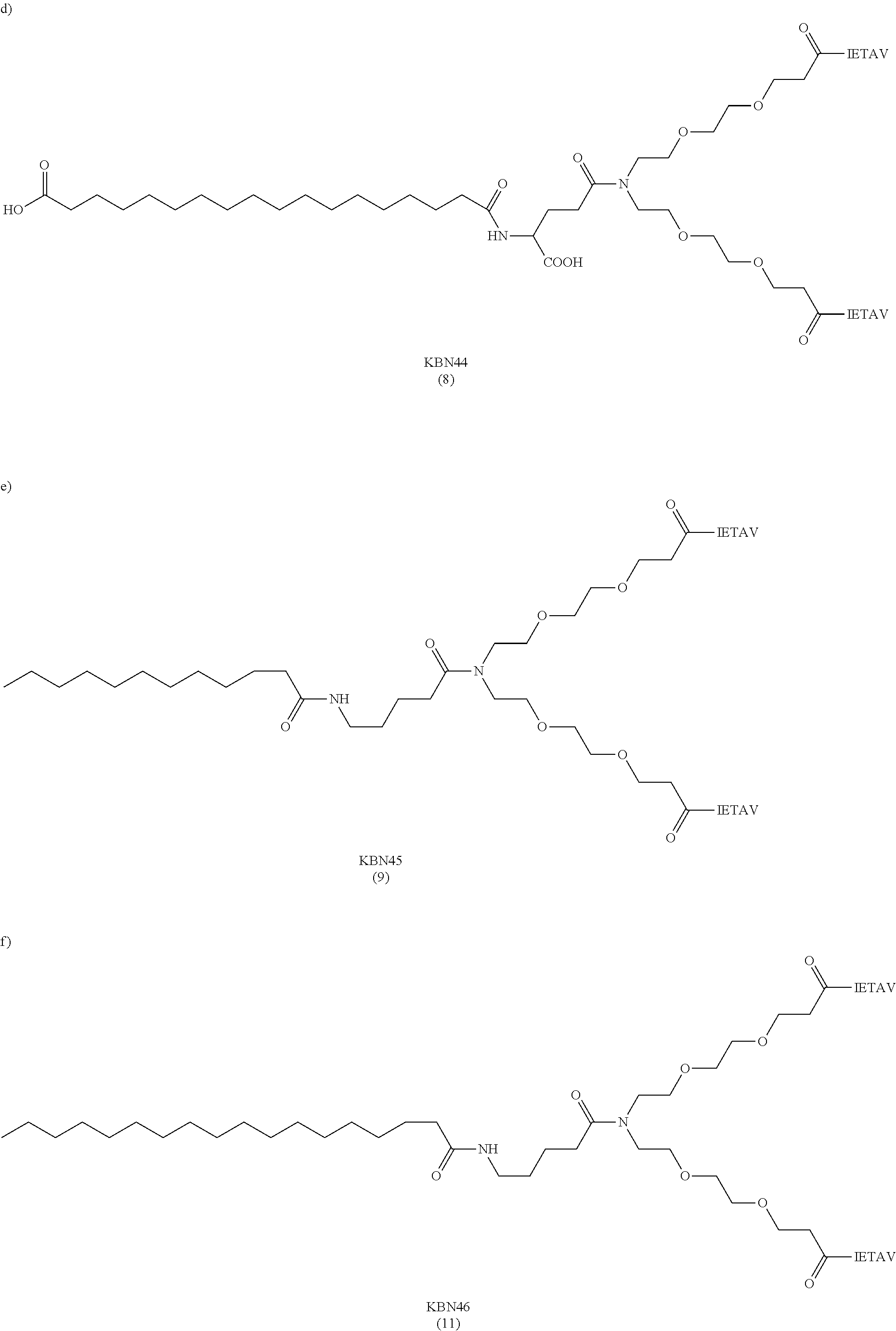

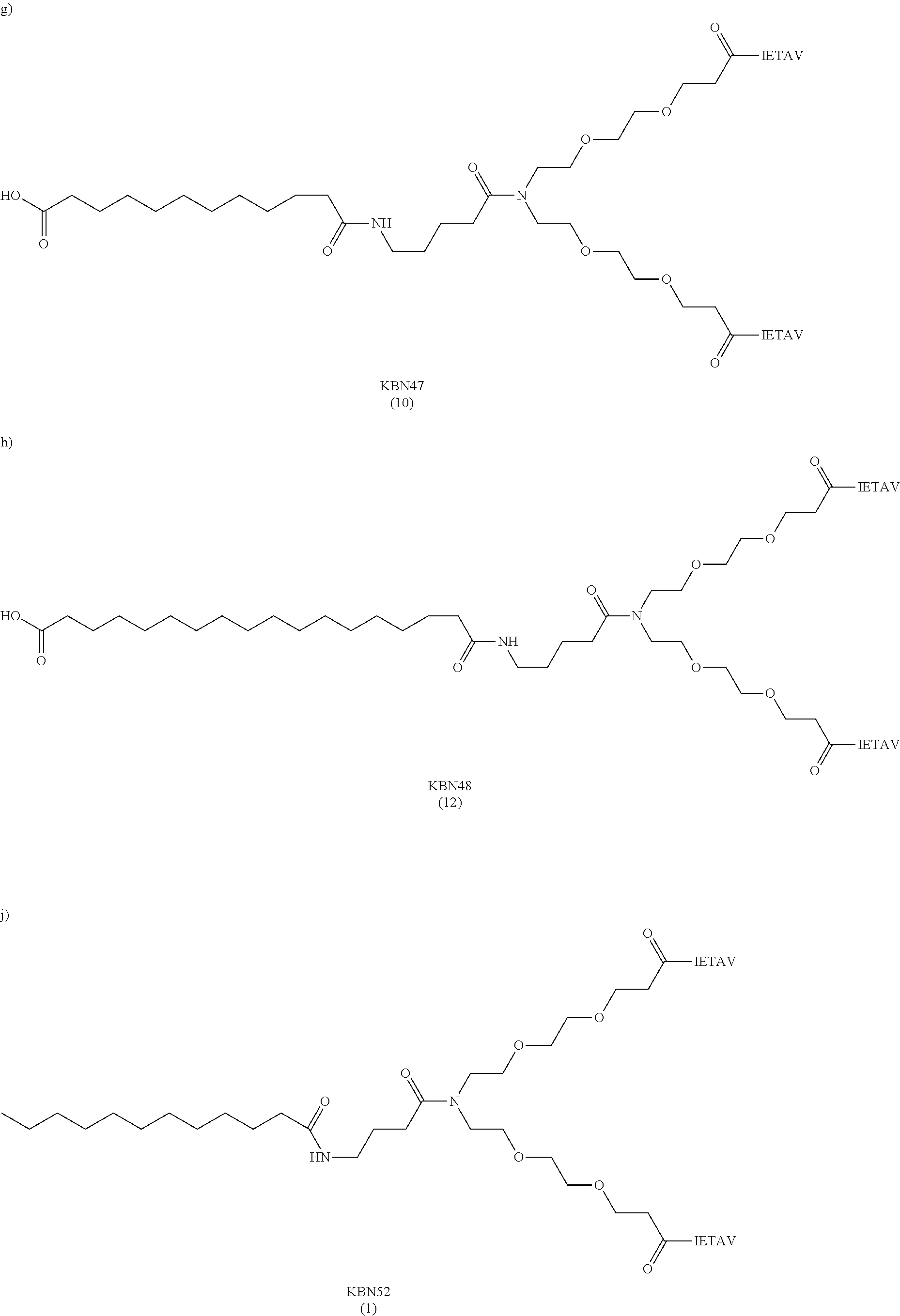

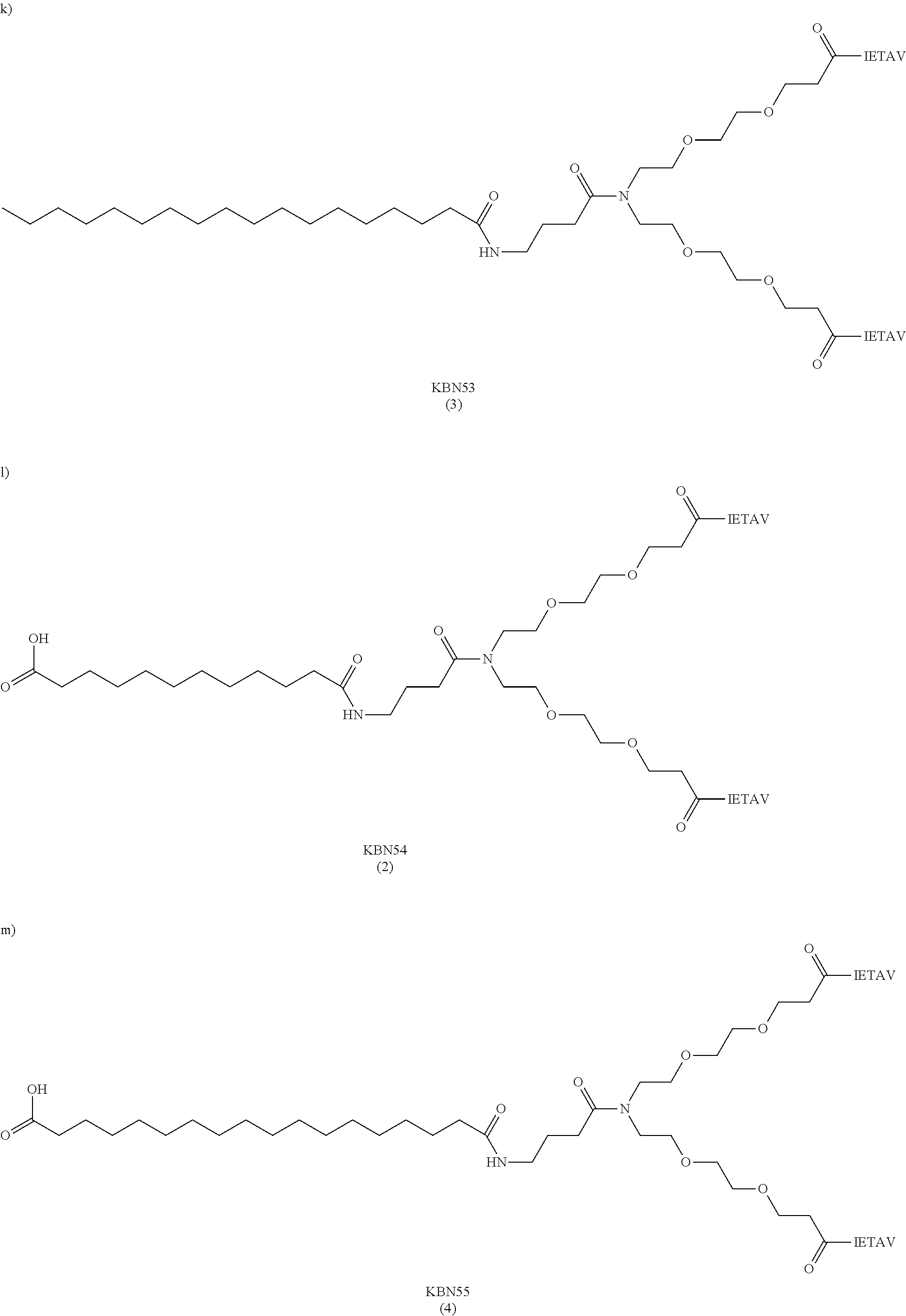

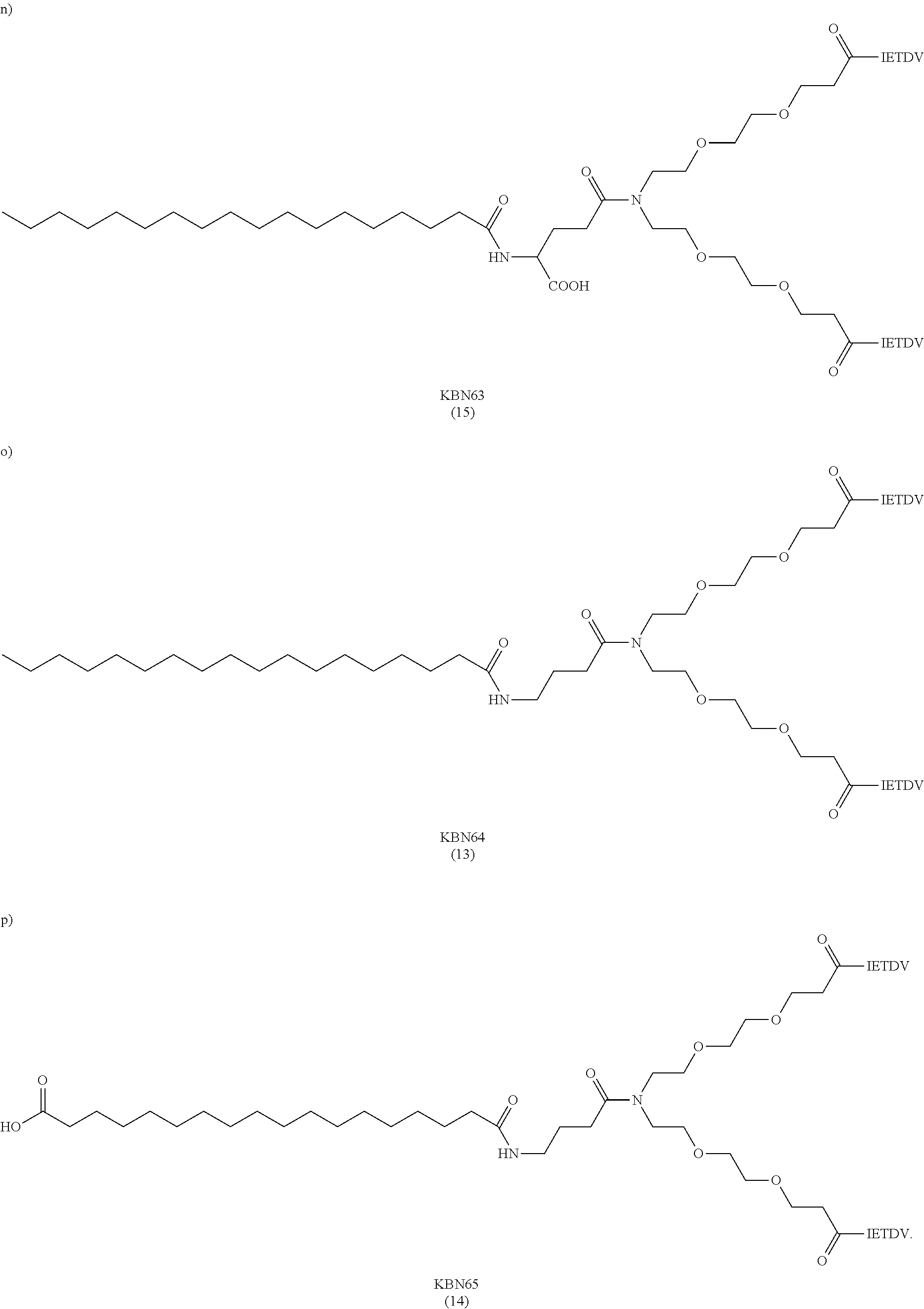

41. The compound according to any one of the preceding claims, in the form of a pharmaceutically acceptable salt or prodrug of said compound.

42. A compound according to any one of the preceding claims for use as a medicament.

43. A compound according to any one of claims 1 to 41 for use in the treatment or prophylaxis of pain.

44. A compound according to any one claims 1 to 41 for use in the treatment or prophylaxis of an excitotoxic-related disease.

45. The compound according to claim 44, wherein the disease is ischemic or traumatic injury to/in/of the CNS.

46. A method of manufacturing the compound according to any one of claims 1 to 41, said method comprising the steps of:
a) preparing a Ns-NPEG diacid linker,
b) preparing a peptide using Fmoc-based solid-phase peptide synthesis,
c) dimerizing Fmoc-deprotected peptide with Ns-NPEG diacid linker
d) coupling a fatty acid to the linker-dimer conjugate, optionally via an intermediate linker, such as an amino acid linker (L₂)

47. The method according to claim 46 (step a), wherein the ortho-nitrobenzenesulfonyl (Ns)-protected NPEG linker is produced on solid-phase or in solution.

48. The method according to claim 47, wherein the solid-phase procedure is performed by loading a solid support suitable for solid-phase peptide synthesis, such as 2-chlorotrityl chloride resin, with Fmoc-NH-PEG-CH₂CH₂COOH, using an organic solvent such as DCM, DMF, ACN or THF; and a base such as DIPEA, DBU, collidine or NMM.

49. The method according to any one of claims 47 to 48 wherein the Fmoc group is removed by a base such as piperidine, dimethylamine, morpholine, piperazine, dicyclohexylamine or DMAP) in a suitable solvent such as DMF, DCM, ACN, THF.

50. The method according to claims 47 to 49 wherein the ortho-nitrobenzenesulfonyl chloride is coupled to the free amine using a suitable base such as DIPEA; and a suitable solvent such as THF, DCM thus obtaining Ns-NH-PEG-CH₂CH₂COO-Resin.

51. The method according to according to claims 47 to 50 wherein the second part of the linker product is connected to the resin-bound linker-part using Mitsunobu-chemistry, and wherein the resin subsequently is treated with triphenylphosphine, HO-PEG-CH₂CH₂COOtBu, solvent, and ester- or amide reagents of azodicarboxylic acid such as diisopropyl azodicarboxylate (DIAD), diethyl azodicarboxylate (DEAD) or 1,1′-(Azodicarbonyl)-dipiperidine (ADDP); and subsequently treating the resin with acid, such as trifluoroacetic acid (TFA), thus obtaining the final Ns-NPEG diacid linker.

52. The method according to claim 46 (step a), wherein the solution-phase procedure is performed by protecting the amine group of NH₂—PEG-CH₂CH₂COOtBu with Ns, followed by Mitsunobu chemistry in solution using triphenylphosphine and DIAD, DEAD, or ADDP, or similar reagents, HO-PEG-CH₂CH₂COOtBu, and a suitable solvent (THF, DCM), and treating with acid, such as TFA, thus obtaining Ns-protected NPEG-linker.

53. The method according to claim 46 (step b), wherein the peptide is synthesized using Fmoc-based solid-phase peptide synthesis using a solid support, such as 2-chlorotrityl chloride resin or Wang resin, Fmoc-protected amino acids, base, coupling reagents such as HBTU [N,N,N′,N′-Tetramethyl-O-(1H-benzotriazol-1-yl)uronium hexafluorophosphate], O-(7-azabenzotriazol-1-yl)-N,N,N′,N′-tetramethyluronium hexafluorophosphate [HATU], PyBOB, DIC/HOBt) and solvents; or alternatively by use of activated ester of Fmoc-protected amino acids such as pentafluorophenyl, succinimide.

54. The method according to claim 46 (step c) wherein the Fmoc-deprotected resin-bound peptide is dimerized with the Ns-NPEG diacid linker using an on-resin dimerization process comprising repetitive treatments of the resin with the Ns-NPEG diacid linker in sub-stoichiometric amounts such as 1/6, base, coupling reagent, and suitable solvents such as DMF, DCM or THF; or alternatively by use of activated esters, such as pentafluorophenyl or succinimide, of the Ns-NPEG linker.

55. The method according to claim 46 (step c), wherein the dimerization process is performed in solution using either the activated ester such as pentafluorophenyl or succinimide of the Ns-NPEG linker together with 1-hydroxy-7-azabenzotriazole (HOAt) or hydroxybenzotriazole (HOBt) and suitable side chain-protected peptide such as tert-butyl; in a solvent such as ACN, DMF, DCM, or THF.

56. The method according to claim 46 (step c), wherein the dimerization process is performed in solution by using the Ns-NPEG diacid linker, coupling reagents (e.g. HBTU, HATU etc), base and solvents.

57. The method according to any one of claims 54 to 56, further comprising the step of removing the Ns-group by thiols, such as mercaptoethanol and 1,8-diazabicyclo[5.4.0]undec-7-ene (DBU) or by sodium thiophenolate.

58. The method according to claims 46 (step d), further comprising coupling the fatty acid to the linker-dimer conjugate using coupling reagent and base for activation, or using activated esters.

59. The method according to claim 46 (step d), further comprising coupling the amino acid linker to the free nitrogen of the NPEG-dimerized and resin-bound peptide by consecutive couplings of the Fmoc-protected linker such as Fmoc-Glu-OtBu, Fmoc-GABA or Fmoc-5-Ava-OH; by using coupling reagent and base for activation or by activated ester of Fmoc-protected amino acid linkers.

60. The method according to any one of claims 58 to 59, wherein the carboxylic group of the fatty acid optionally is protected as esters, such as a methyl ester.

61. The method according to any one of claims 46 to 60 wherein the fatty acid-linked dimeric ligands are optionally cleaved from the resin using concomitant side-chain deprotection by acids such as TFA or HCl.

62. The method according to any one of claims 46 to 61, wherein ester protection groups are removed by stirring the cleaved products in aqueous base such as NaOH or LiOH; followed by acidification using TFA or HCl.

63. The method according to any one of claims 46 to 62 wherein the final product is obtained by lyophilization and purification using chromatographic methods.

64. The method according to claim 63, wherein the purification is performed using HPLC.


SEQUENCE LISTING (6 sequences)

SEQ ID NO: 1 - protein, 4 aa, Artificial Sequence
  X is independently any amino acid
XXXX

SEQ ID NO: 2 - protein, 4 aa, Artificial Sequence
  X is independently any amino acid
XXXX

SEQ ID NO: 3 - protein, 5 aa, Artificial Sequence
  specific peptide
IETAV

SEQ ID NO: 4 - protein, 5 aa, Artificial Sequence
  specific peptide
IETDV

SEQ ID NO: 5 - protein, 5 aa, Artificial Sequence
  X is independently any amino acid
XXXXX

SEQ ID NO: 6 - protein, 5 aa, Artificial Sequence
  X is independently any amino acid
XXXXX